(19) United States
(12) Patent Application Publication
(10) Pub. No.: US 2022/0008276 A1
(43) Pub. Date: Jan. 13, 2022

(54) Pressure Differential-, Ultrasound-, and Magnetic-Based Methods for Treating Viral Infections and other Pathogenic Diseases, Sterilizing Medical Equipment, and Enhancing Fatty Tissue Reduction

(71) Applicants: Gabriel Curiel, Torre (MX); Gordon G. Waggett, Houston, TX (US); John R. Klug, Denver, CO (US)

(72) Inventors: Gabriel Curiel, Torre (MX); Gordon G. Waggett, Houston, TX (US); John R. Klug, Denver, CO (US)

(21) Appl. No.: 17/369,803

(22) Filed: Jul. 7, 2021

Related U.S. Application Data
(60) Provisional application No. 63/049,607, filed on Jul. 8, 2020.

(51) Int. Cl. A61G 10/02; A61B 17/22; A61N 2/00; A61M 15/00; A61L 2/02

(52) U.S. Cl. CPC A61G 10/026; A61B 17/22004; A61N 2/004; A61M 15/00; A61L 2/02; A61L 2202/24; A61M 2202/02; A61M 2202/0007; A61G 2220/10; A61G 2200/10; A61B 2017/22007

(57) ABSTRACT

The present disclosure is directed to methods of treating subject patients (such as humans, animals, plants) suffering from a pathogenic disease such as COVID-19 in humans, via the administration of pressure changes in the patient sufficient to cause a pressure differential to be created between the inside and outside of the outer membrane or envelope of the pathogen thereby destroying or disabling the pathogen. In one embodiment, a hyperbaric chamber is used to administer pressure increases and/or pressure decreases to create such pressure differential. The hyperbaric chamber could comprise a single user or multi-user unit, a pressurized body suit, or the pressurizable fuselage of an aircraft. In another embodiment, patients are placed in an aircraft, and the cabin pressure, while on the ground, or in flight, is adjusted upwardly or downwardly to create such pressure differential. The pressure differential methods can also include the use of external gases to enter the patient's body and/or lungs to facilitate disruption of the pathogen outer membrane as well as application of variations in temperature and/or humidity. A mobile treatment unit is also disclosed. Also disclosed are methods of using ultrasonic cavitation or MRI (or other sonic or magnetic field forces sufficient to disrupt the functionality of the pathogen), or a combination of ultrasound and MRI on the exterior of a patient in a desired anatomical region of the patient to assist with the destruction or disabling of a pathogen infecting that anatomical region of the patient, e.g., the patient's lungs, said method being employed at ambient pressures or in an increased or decreased pressure environment created within a hyperbaric chamber. The ultrasound and/or MRI methodologies could also be used to treat other pathogenically afflicted areas of the patient's body. Additionally, a pressure-differential method of sterilization of medical equipment is disclosed employing a hyperbaric or other pressure or vacuum chamber. Also disclosed is an enhanced method of nonsurgical fat reduction in humans by employing ultrasonic cavitation within a hyperbaric chamber, including the use of HBOT therapies. Furthermore, the use of these methodologies and systems have application in treatment of patients post-infection and in other areas of medicine and health, such as for example, treating wounds, the effects of aging, inflammation, and the effects of other maladies.

### CROSS-REFERENCE TO RELATED APPLICATIONS

This application claims the benefit of the filing date of and priority to: U.S. Provisional Application Ser. No. 63/049,607 entitled “Pressure Differential-Based Methods for Treating Viral Infections, Sterilizing Medical Equipment, and Enhancing Fatty Tissue Reduction” and filed Jul. 8, 2020, Confirmation No. 3719; said provisional application is incorporated by reference herein in its entirety for all purposes.

### STATEMENT REGARDING FEDERALLY SPONSORED RESEARCH OR DEVELOPMENT

Not Applicable.

### BACKGROUND OF THE INVENTION

### Field of the Present Disclosure

The present disclosure relates generally to treatment of humans (or animals, including pets and livestock, beneficial insects including pollinator bees, plant life and foodstock) infected with viruses, or afflicted with other infectious or pathogenic diseases. As a particularly relevant example, the present disclosure relates also to methods of treatment of humans infected with a novel coronavirus and its variants both during and post-infection. The present disclosure also relates to a mobile method of treating one or more subject patients infected with a pathogen (e.g., a virus, such as a novel coronavirus and its variants), and a mobile treatment unit for treating subject patients infected with a pathogen, (e.g., a virus, such as a novel coronavirus and its variants).

Additionally, the present disclosure is directed to new nondestructive methods for disinfecting medical equipment, including surgical masks. to permit ready reuse without significant loss of functionality.

Further, the present disclosure relates generally to enhanced fatty tissue reduction procedures.

### Background

Over the past two decades, the world has experienced serious infectious disease outbreaks such as Ebola, Influenza A (H1N1), SARS, MERS, Zika and now the novel coronavirus 2019 (SARS-CoV-2) that causes COVID-19. These viruses have had a massive global impact in terms of economic disruption, including the strain on local and global public health. The severity and importance of the worldwide Coronavirus pandemic is well established. Although vaccine development and deployment is one strategy that must be followed to help to eliminate the virus, such development and deployment takes time and does not address those patients who are currently suffering from the active viral infection. This current Coronavirus pandemic has already shown the introduction of mutations of the virus—mutations that may or may not be effectively dealt with by the initial vaccine developments. Further, other viral outbreaks can happen in the future—thus, there remains a need to address and treat patients rapidly when a pandemic breaks out, and to make such treatments available on very large scale. One additional ramification of the global pandemic is that certain patients who have survived the active viral infection nonetheless are suffering from a number of post-infection complications referred to as “post-COVID syndrome” or “Covid long haul”. Thus, there likewise remains a need for these treatments to extend beyond the active viral infection stages and assist those dealing with post-COVID syndrome.

Likewise, pathogenic disease (whether naturally occurring or instigated as a form of bioterrorism) can cause severe economic harm when the disease affects animals, including pets and livestock, as well as beneficial insects, such as pollinating bees, and on plant life including foodstock plants and crops. For example, the world's bee population is down drastically and bees are known to suffer from at least 22 viruses, 7 viruses of which are threatening world bee keeping: Bee Paralysis (ABPV); Black Queen Cell Virus (BQCV); Chronic Bee Paralysis (CBPV), Deformed Wing Virus (DWV); Israel Acute Paralysis (IAPV); Kashmir Bee Virus (KBV) and Sacbrood Virus (SBV). Therefore, there remains a need to address these pathogenic diseases in subject patients, including humans, animals, plants, insects and foodstock.

Humans infected with viruses are often treated with a number of antiviral drug treatment regimens. However, when a novel virus, such as the novel coronavirus (officially referred to as SARS-CoV-2, and previously referred to as 2019-nCoV) creates a pandemic outbreak of the coronavirus disease 2019 (officially referred to as COVID-19), it introduces challenges within the medical community for finding rapid treatment methodologies that can assist in treating a patient infected with such virus. While the disclosure herein is generally directed to pathogens, specific disclosure is provided directed to, e.g., the novel coronavirus pathogen and its variants (including alpha-, delta-, etc.) Thus, the teachings herein have application to pathogens, including generally, viruses, bacteria, fungi, parasites, protozoa, helminths and worms, and including for example, the specifically identified pathogens such as the novel coronavirus and its mutations/variants.

Boopathi et al. provide useful information about the novel 2019 coronavirus structure and mechanism of action for use in structure-based computational simulations to aid in screening of known drugs for use in combating the coronavirus. It is well understood that the novel coronavirus contains viral surface proteins, spike glycoprotein, envelope and membrane that are embedded in a lipid bilayer. A single-stranded positive-sense viral RNA is associated with the nucleocapsid protein. The coronavirus spike (S) protein attaches to angiotensin converting enzyme 2 (ACE2) receptors found on the surface of many human cells, including those in the lungs allowing virus entry. See Boopathi, Subramanian et al. “Novel 2019 coronavirus structure, mechanism of action, antiviral drug promises and rule out against its treatment.” J Biomolecular Structure & Dynamics. vol. 39,9 (2021): 3409-3418. doi:10.1080/07391102.2020.1758788.

According to Thibodeaux et al., “[p]atients with COVID-19 disease present with a wide array of symptoms, ranging from mild flu-like complaints to life threatening pulmonary and cardiac complications. Older people and patients with underlying disease have an increased risk of developing severe acute respiratory syndrome (SARS) requiring mechanical ventilation. Once intubated, mortality increases exponentially. A number of pharmacologic regimens, including hydroxychloroquine-azithromycin, antiviral therapy (e.g, Remdesevir), and anti-IL-6 agents (e.g., Toclizumab), have been highlighted by investigators over the course of the pandemic, based on the therapy's potential to interrupt the viral life-cycle of SARS-CoV-2 or preventing cytokine storm. At present, there have been no conclusive series of reproducible randomized clinical trials demonstrating the efficacy of any one drug or therapy for COVID-19. See Thibodeaux K, Speyrer M, Raza A, Yaakov R, Serena T E. Hyperbaric oxygen therapy in preventing mechanical ventilation in COVID-19 patients: a retrospective case series. J Wound Care. 2020 May 1; 29(Sup5a):S4-S8. doi: 10.12968/jowc.2020.29.Sup5a.S4. PMID: 32412891.

The Mayo Clinic teaches that Hyperbaric Oxygen Therapy (“HBOT”)(100% oxygen in a pressurized room or tube) is a well-established treatment for decompression sickness, such as that which can result from scuba diving. Other conditions treated with hyperbaric oxygen therapy include serious bacterial infections, bubbles of air in a patient's blood vessels, and wounds that will not heal as a result of diabetes or radiation injury. Hyperbaric oxygen therapy enhances the level of oxygen in the blood to help fight bacteria and stimulate the release of growth factors and stem cells to promote healing. During treatment by hyperbaric oxygen therapy, the air pressure in the room is about two to three times normal air pressure and can cause a pressure discomfort in the inner ears similar to what one might feel in an airplane or at a high elevation. Hyperbaric chambers work by increasing the pressure outside the body, and they are commonly used to treat breathing-related conditions. Under this pressure, the lungs work less to breathe because the air pressure is so much greater that air forces itself in. Further, at higher pressures, oxygen is more soluble, so every breath gets more oxygen into the bloodstream. Higher levels of blood oxygenation, according to the Mayo Clinic, can promote healing and fight infection. See Mayo Clinic, “Hyperbaric oxygen therapy”, www.mayoclinic.org/tests-procedures/hyperbaric-oxygen-therapy/about/pac-20394380 (last accessed 6/27/2020). Although the Mayo Clinic proposes the use of hyperbaric oxygen therapy to treat various medical conditions, including bacterial infections, there is no suggestion that such therapy be used to treat a viral infection.

Further, HBOT has been suggested for use in the treatment of aging. Hachmo et al. report that the aging process is “characterized by the progressive loss of physiological capacity. At the cellular level, two key hallmarks of the aging process include telomere length (TL) shortening and cellular senescence. Repeated intermittent hyperoxic exposures, using certain hyperbaric oxygen therapy (HBOT) protocols, can induce regenerative effects which normally occur during hypoxia.” This biological deterioration is considered “a major risk factor for cancer, cardiovascular diseases, diabetes and Alzheimer's disease among others.” Hachmo et al. set out to evaluate whether HBOT affects TL and senescent cell concentrations in a normal, non-pathological, aging adult population. Their recent study indicates that hyperbaric oxygen treatments (HBOT) in healthy aging adults can stop the aging of blood cells and reverse the aging process and illnesses associated with aging. In the biological sense, the adults' blood cells actually grow younger as the treatments progress. In particular, “[r]epeated intermittent [or pulsed] hyperoxic exposures, using certain HBOT protocols, can induce physiological effects which normally occur during hypoxia in a hyperoxic environment, the so called hyperoxic-hypoxic paradox.” These HBOT interventions included having the patients breathe 100% oxygen by mask at 2ATA for 90 minutes with 5-minute air breaks every 20 minutes. See, Hachmo, Yafit, et al. “Hyperbaric Oxygen Therapy Increases Telomere Length and Decreases Immunosenescence in Isolated Blood Cells: a Prospective Trial.” Aging, Nov. 18, 2020, doi:10.18632/aging.202188. See also American Friends of Tel Aviv University. “Hyperbaric oxygen treatment: Clinical trial reverses two biological processes associated with aging in human cells.” ScienceDaily. 11/20/2020. <www.sciencedaily. com/releases/2020/11/201120150728.htm>. See also Leichman A K, Study shows hyperbaric oxygen can reverse the aging process. Israel21c. 11/22/2020 https://www. israel21c.org/an-israeli-doctors-prescription-for-premium-peanut-butter/(accessed 7/4/2021); Leichman A K, From aging to chronic wounds, is hyperbaric oxygen a cure-all? Israel21c. 01/05/2021 https://www.israel21c.org/from-aging-to-chronic-wounds-can-hyperbaric-oxygen-cure-everything/(accessed 6/12/2021).

HBOT has also been shown to induce the generation of new blood vessels (angiogenesis) in different body organs, and in one study, to recover erectile function in men. Hadanny, A., Lang, E., Copel, L. et al. Hyperbaric oxygen can induce angiogenesis and recover erectile function. Int J Impot Res 30, 292-299 (2018). https://doi. org/10.1038/s41443-018-0023-9. Hadanny et al. also discuss that HBOT incorporates the inhalation of 100% oxygen at pressures exceeding 1 atmosphere absolute (ATA), thus increasing the amount of oxygen dissolved in the body tissues (citing Fosen K M, Thom S R. Hyperbaric oxygen, vasculogenic stem cells, and wound healing. Antioxid Redox Signal. 2014; 21:1634-47). Hadanny et al. indicate that one of the most interesting mechanisms induced by HBOT is angiogenesis mediated by release of omnipotent stem cells capable of differentiating into endothelial cells (citing Thom S R, Bhopale V M, Velazquez O C, Goldstein L J, Thom L H, et al. Stem cell mobilization by hyperbaric oxygen. Am J Physiol Heart Circ Physiol. 2006; 290:H1378-86; Hopf H W, Gibson J J, Angeles A P, Constant J S, Feng J J, et al. Hyperoxia and angiogenesis. Wound Repair Regen. 2005; 13:558-64; and Peng Z R, Yang A L, Yang Q D. The effect of hyperbaric oxygen on intracephalic angiogenesis in rats with intracerebral hemorrhage. J Neurol Sci. 2014; 342:114-23). Hadanny et al. also report that HBOT boosts the release of vascular endothelial growth factor (VEGF) and hypoxia-inducible factor-1alpha (HIF-1alpha), crucial mediators for the angiogenesis process (citing Peng et al.). In addition, they discuss that improved oxygenation by HBOT creates the necessary environment needed for stem cells proliferation and that pre-clinical and clinical studies have demonstrated that HBOT can induce angiogenesis even in tissues with low regenerative potential such as the brain (citing Chen J, Zhang Z G, Li Y, Wang L, Xu Y X, et al. Intravenous administration of human bone marrow stromal cells induces angiogenesis in the ischemic boundary zone after stroke in rats. Circ Res. 2003; 92:692-9; Jiang Q, Zhang Z G, Ding G L, Zhang L, Ewing J R, et al. Investigation of neural progenitor cell induced angiogenesis after embolic stroke in rat using MRI. Neuroimage. 2005; 28:698-707; and Tal S, Hadanny A, Berkovitz N, Sasson E, Ben-Jacob E, et al. Hyperbaric oxygen may induce angiogenesis in patients suffering from prolonged post-concussion syndrome due to traumatic brain injury. Restor Neurol Neurosci. 2015; 33:943-51).

The food industry employs High Pressure Processing (HPP) as a means for cold pasteurization of food products. High Pressure Processing (HPP) is a cold pasteurization technique by which products, already sealed in its final package, are introduced into a vessel and subjected to a high level of isostatic pressure (300-600 MPa/43,500-87,000 psi) transmitted by water. Pressures above 400 MPa/58,000 psi at cold (+4° C. to 10° C.) or ambient temperature inactivate the vegetative flora (bacteria, virus, yeasts, molds and parasites) present in food, extending the products shelf life importantly and guaranteeing food safety. See Hiperbaric, S.A., “HPP Technoology”, www.hiperbaric.com/en (last accessed 6/27/2020). High Pressure Processing (HPP) is based on the Le Chatelier principle which states that actions that have a net volume increase will be retarded and actions that have a net volume decrease will be enhanced. HPP utilizes isostatic or hydrostatic pressure which is equal from every direction. During HPP, foods are subjected to pressures up to 100,000 psi. which destroy pathogenic microorganisms by interrupting their cellular functions. Within a living bacteria cell, many pressure sensitive processes such as protein function, enzyme action, and cellular membrane function are impacted by high pressure resulting in the inability of the bacteria to survive. See HPD, Inc. (Cincinnati, Ohio) “What is High Pressure Pasteurization?”, www.highpressuredynamics.com/id3.html (last accessed 6/27/2020). Although this HPP technique is an effective mechanism to inactivate viruses in packaged foods, these pressures cannot be used to treat humans.

HPP is based on two basic principles: Le Chatelier's principle and Isostatic principle (Pascal's Law). The Le Chatelier's principle states that if a change in conditions is applied on a system in equilibrium, then the system will try to counteract that change and restore the equilibrium. The Isostatic principle states that the HPP is volume-independent; therefore, pressure is transmitted instantaneously and uniformly throughout a sample, and pressure gradients do not exist, so that the size and geometry of the product is irrelevant. Typically, HPP of food is carried out at 300-600 Mega Pascals (MPa) at room temperature for 2-30 min. The HPP is an alternative to thermal processing as it is operated at ambient temperatures, ensuring little or no heat induced sensory changes in its components of food. It is because of the fact that, the smaller organic molecules responsible for colors, flavors, and nutrients (e.g., vitamins) have covalent bonding dominantly or exclusively, which are hardly affected by HPP. Whereas, the large bio-molecules such as proteins, nucleic acids and polysaccharides that depend on non-covalent bonding (like hydrophobic interactions, hydrogen bonds, etc.) to maintain structure and function are most affected.

Similar to the use of hyperbaric conditions for reducing the microbial burden of foodstuffs, U.S. Pat. No. 9,169,302 (Carboulec et al.) relates to hyperbaric devices (using pressures between about 3000 bars to 5000 bars) for inactivating microorganisms and viruses while retaining their immunogenicity and for making and producing the soluble, disaggregated, refolded or active immunogenic or therapeutic proteins from inclusion bodies produced from prokaryotes or eukaryotes. The work of Carboulec et al. encompasses hyperbaric methods for inactivating pathogenic organisms, and methods for producing vaccine compositions using the inactivated pathogens. The hyperbarically inactivated microorganisms are safer and more immunogenic than chemically inactivated microorganisms. Similarly, the solubilized proteins have superior properties compared to more heavily aggregated proteins, including reduced non-specific immune reactions.

Grau-Bartual, et al., suggest that studies of lung supportive devices employing noninvasive positive pressure airway ventilation appear to indicate that applying pressure on the epithelial cells using CPAP (continuous positive airway pressure) indicates that cells can survive the 20 cm H2O pressure for 10 minutes and that the cell layer integrity remains intact. However, TEER (trans-epithelial electrical resistance) measurements showed that the cell layer is less permeable after pressure application, which suggest a possible compression effect also indicated in previous literature. Grau-Bartual, et al., “Effect of positive airway pressure oscillations on human epithelial cells”, Vibroengineering PROCEDIA, Vol. 24, 2019, p. 58-62. www.jvejournals.com/article/20756 (last accessed 6/27/2020). Also it is known that CPAP compresses human respiratory epithelial cells, reduces their permeability and mucus secretion rate, dries the airway surface liquid layer, and produces an inflammatory response. See Grau-Bartual, et al., “Effect of continuous positive airway pressure treatment on permeability, inflammation and mucus production of human epithelial cells”, ERJ Open Research 2020 6: 00327-2019, openres.ersjournals. com/content/6/2/00327-2019, last accessed 6/27/2020. These studies appear to suggest that factors associated with CPAP use (and hence application of pressure on the epithelial cells in the lungs) may disturb the natural mechanism in the respiratory system to restore the ASL (airway surface liquid) layer properties, such as the mucus/water ratio.

Currently, COVID-19 patients experiencing acute respiratory distress syndrome (ARDS) are administered oxygen via an endotracheal tube/ventilator in an ICU unit. It is not recommended to use traditional high pressure nasal cannula, oxygen masks, or BiPAP masks (non-invasive positive pressure ventilation (NIPPV)) on a COVID-19 patient due to the possible aerosolization of the virus with the consequent risk to medical personnel. As an alternative to the use of traditional NIPPV masks, bubble helmets are available, e.g., from Sea-Long Medical Systems, LLC (Waxahachie, Tex.) to enclose and secure around the patient's head and neck to administer the oxygen via attachment to fresh gas flow or to a ventilator. The non-invasive bubble helmet mask technique is thought to reduce the number patients requiring intubation. One such helmet device is disclosed in U.S. Pat. No. 9,931,482 (Ritchie et al.).

Masson et al. provide a literature review in the field of high-pressure biotechnology and report that viruses are very sensitive to moderate pressures between 1 and 3 kbar. As such, inactivation of numerous viruses such as herpes viruses and immunodeficiency viruses by pressure has been successful in connection with, e.g., development of vaccines or other biological materials requiring inactivation of the virus. Masson et al. note as possible applications of pressure the inactivation of viruses in biological compounds, sterilization of biological materials, blood and tools, as well as decreasing the AIDS virus concentration in blood via ex vivo pressure treatment of the blood. See Masson P., Tonello C., Balny C. High-pressure biotechnology in medicine and pharmaceutical science. J. Biomed. Biotechnol. 2001; 1:85-88. doi: 10.1155/S1110724301000158. Similarly, Dumard, et al. report that high hydrostatic pressure (HHP) may be a valuable tool for vaccine development as it promotes viral inactivation. See Dumard C H, Barroso S P C, Santos A C V, Alves N S, Couceiro J N S S, Gomes A M O, Santos P S, Silva J L, Oliveira A C. Stability of different influenza subtypes: How can high hydrostatic pressure be a useful tool for vaccine development? Biophys Chem. 2017 December; 231:116-124. doi: 10.1016/j.bpc.2017.04.002. Epub 2017 Apr. 6. PMID: 28410940. See also Jurkiewicz E, Villas-Boas M, Silva J L, Weber G, Hunsmann G, Clegg R M. Inactivation of simian immunodeficiency virus by hydrostatic pressure. Proc Natl Acad Sci USA. 1995 Jul. 18; 92(15):6935-7. doi: 10.1073/pnas.92.15.6935. PMID: 7624347; PMCID: PMC41445. Given that 1 atmosphere is 0.4 kbar, one can conclude that viruses are very sensitive between 2.5-7.5 atmospheres—well withing human tolerance.

U.S. Pat. No. 10,376,578 (Agrawal) discusses compositions and methods for treating Crohn's disease and related conditions and infections including the use of Hyperbaric Oxygen Treatment (HBOT). The conditions for use of HBOT include administering the medical use of oxygen at a level higher than atmospheric pressure. In one embodiment of this HBOT, the medical use of oxygen at a level higher than atmospheric pressure is employed, for example, at a pressure of about 100% oxygen, or between about 90% and 100% oxygen, at 2.5 atm absolute, or between about 2 and 3 atm absolute, for about 90 minutes or between about 1 hour and 2 hours per session or treatment. In another embodiment, a “triple combination” therapy of this invention, including e.g., hyperbaric oxygen, anti-TNF.alpha. medication and anti-MAP medication, is particularly useful in healing a Crohn's related fistulae. In this treatment regimen, the use of HBOT targets anaerobic bacteria colonizing CD fistulae. Previously, HBOT is predominantly used for accelerating the healing of tissue necrosis (e.g., diabetic foot ulcers) and sports injuries. Agrawal's studies demonstrated that use of this “triple combination” therapy comprising use of the three fistulae therapies Infliximab, HBOT and Anti-MAP (all of which are only partially effective when used alone) resulted in much higher rates of healing.

Research on COVID-19 is advancing at a rapid pace, and many treatment methodologies, including repurposing older drug therapies, have emerged. One preliminary non-peer reviewed study currently indicates neutralization of the SARS-COV-2 by destroying the prefusion Spike conformation as a means of interfering with the ability of the virus to attach to the host cell membrane. Huo, J., et al. (2020), “Neutralization of SARS-CoV-2 by Destruction of The Prefusion Spike”, bioRxiv preprint, biorxiv.org/content/10.1101/2020.05.05.079202v1, last accessed 6/27/2020. See also Huang, Y., Yang, C., Xu, X f. et al. Structural and functional properties of SARS-CoV-2 spike protein: potential antivirus drug development for COVID-19. Acta Pharmacol Sin 41, 1141-1149 (2020). https://doi.org/10.1038/s41401-020-0485-4, noting that further understanding of the structure and function of SARS-CoV-2 S will allow for additional information regarding invasion and pathogenesis of the virus to aid in the design of vaccines and antiviral therapeutics.

Ottawa Hospital Research Institute recently suggested proposing the use of HBOT to treat COVID-19 pneumonia as a means to avoid having to place the patient on a ventilator, and will undertake studies. Ottawa Hospital Research Institute, “Hyperbaric oxygen therapy for COVID pneumonia”, Jun. 26, 2020, www.ohri.ca/newsroom/story/view/1257?1=en#:˜:text=Dr.%20Sylvain%20Boet%20and%20his%20colleagues%20believe% 20that,or%20chamber%20so%20they%20can%20breathe%20100%25%20oxygen, last accessed 6/28/2020. See also C. Fife, “Hyperbaric Oxygen Therapy for Severe COVID-19 Pneumonia?”, Apr. 4, 2020, carolinefifemd.com/2020/04/04/hyperbaric-oxygen-therapy-for-severe-covid-19-pneumonia/, last accessed 7/7/2020 (suggesting the potential use of HBOT therapy for severe COVID-19 pneumonia and hypoxia); Hadanny et al., NIH, “Hyperbaric Oxygen Therapy Effect in COVID-19 RCT (HBOTCOVID19) (HBOTCOVID19)”, clinicaltrials.gov/ct2/show/NCT04358926, last accessed 7/7/2020 (proposed clinical trial to evaluate the efficacy of HBOT in moderate-severe COVID-19 patients in a randomized controlled manner to evaluate proposed known physiological effects of HBOT, namely, increased competitive binding of oxygen to the hemoglobin molecule, tissue oxygenation, and anti-inflammatory properties).

Also, the safety and efficacy of hyperbaric oxygen for COVID-19 patients with respiratory distress was studied by Gorenstein et al. This was a single-center clinical trial of COVID-19 patients at NYU Winthrop Hospital from March 31 to Apr. 28, 2020. Patients in this trial received hyperbaric oxygen therapy at 2.0 atmospheres of pressure in monoplace hyperbaric chambers for 90 minutes daily for a maximum of five total treatments. Controls were identified using propensity score matching among COVID-19 patients admitted during the same time period. Using competing-risks survival regression, the researchers analyzed their primary outcome of inpatient mortality and secondary outcome of mechanical ventilation. Gorenstein et al. treated 20 COVID-19 patients with hyperbaric oxygen. Ages ranged from 30 to 79 years with an oxygen requirement ranging from 2 to 15 liters on hospital days 0 to 14. Of these 20 patients, two (10%) were intubated and died, and none remain hospitalized. Among 60 propensity-matched controls based on age, sex, body mass index, coronary artery disease, troponin, D-dimer, hospital day, and oxygen requirement, 18 (30%) were intubated, 13 (22%) have died, and three (5%) remain hospitalized (with one still requiring mechanical ventilation). Assuming no further deaths among controls, Gorenstein et al. estimated that the adjusted subdistribution hazard ratios were 0.37 for inpatient mortality (p=0.14) and 0.26 for mechanical ventilation (p=0.046). Gorenstein et al.'s study suggests that though limited by its study design, their results demonstrate the safety of hyperbaric oxygen among COVID-19 patients and strongly suggests the need for a well-designed, multicenter randomized control trial. See, Gorenstein S A, Castellano M L, Slone E S, Gillette B, Liu H, Alsamarraie C, Jacobson A M, Wall S P, Adhikari S, Swartz J L, McMullen J J S, Osorio M, Koziatek C A, Lee D C. Hyperbaric oxygen therapy for COVID-19 patients with respiratory distress: treated cases versus propensity-matched controls. Undersea Hyperb Med. 2020 Third-Quarter; 47(3):405-413. PMID: 32931666.

Similarly, Thibodeaux et al., supra, studied the use of HBOT in the treatment of COVID-19 positive patients (n=5) at a single institution between 13 and 20 Apr. 2020. All the patients had tachypnoea and low oxygen saturation despite receiving high FiO2. HBOT was added to prevent the need for mechanical ventilation. A standard dive profile of 2.0ATA for 90 minutes was employed. Patients received between one and six treatments in one of two dedicated monoplace hyperbaric chambers. Thibodeaux et al., reported that all the patients recovered without the need for mechanical ventilation. Following HBOT, oxygen saturation increased, tachypnoea resolved and inflammatory markers fell. This small sample of patients exhibited dramatic improvement with HBOT. Most importantly, HBOT potentially prevented the need for mechanical ventilation. Larger studies are likely to define the role of HBOT in the treatment of this novel disease.

According to Senniappan et al., although 85% of patients infected with COVID-19 remain asymptomatic, 5% show severe symptoms such as hypoxaemic respiratory failure and multiple end organ dysfunction (MODS) requiring intensive care unit (ICU) admission with a mortality rate of about 2.8%. Since a definitive treatment is yet to be identified, preventive and supportive strategies remain the mainstay of management. Supportive measures such as oxygen therapy with nasal cannula, face mask, noninvasive ventilation, mechanical ventilation and even extreme measures such as extracorporeal membrane oxygenation (ECMO) fail to improve oxygenation in some patients. Hence, hyperbaric oxygen therapy (HBOT) has been proposed as a supportive strategy to improve oxygenation in COVID-19 patients. HBOT is known to increase tissue oxygenation by increasing the amount of dissolved oxygen in plasma. HBOT also mitigates tissue inflammation thus reducing the ill effects of cytokine storm in COVID-19 patients. Though there is limited literature available on HBOT in COVID-19 patients, considering the present need for additional supportive therapy to improve oxygenation, HBOT has been proposed as a novel supportive treatment in COVID-19 patients. According to Senniappan et al., COVID-19 has become a serious threat to humanity despite the currently available advanced medical care. HBOT seems to be a promising supportive therapy with negligible side effects in treating COVID-19 patients. It has the additional advantage of less viral aerosolization compared to other traditional ventilatory strategies used in improving oxygenation. More studies need to be done in this field before it can be recommended for the management of COVID-19 patients. See, Senniappan K, Jeyabalan S, Rangappa P, Kanchi M. Hyperbaric oxygen therapy: Can it be a novel supportive therapy in COVID-19? Indian J Anaesth. 2020 October; 64(10):835-841. doi: 10.4103/ija.IJA_613_20. Epub 2020 Oct. 1. PMID: 33437070; PMCID: PMC7791429.

According to McCallum et al., coronavirus entry into susceptible cells is a complex process requiring concerted action of receptor binding and proteolytic processing of the S protein (the homotrimeric transmembrane S glycoprotein protruding from the viral surface) to promote virus-cell fusion. Studies of the type conducted by McCallum et al. are useful tools for vaccine design, structural biology, serology and immunology studies. See McCallum, M., Walls, A. C., Bowen, J. E. et al. Structure-guided covalent stabilization of coronavirus spike glycoprotein trimers in the closed conformation. Nat Struct Mol Biol 27, 942-949 (2020). https://doi.org/10.1038/s41594-020-0483-8. Recently, Olvera de la Cruz et al. discovered a potential weakness of the SARS-CoV-2 regarding the polybasic cleavage sites, a feature that is unique to the SARS-CoV-2 when compared to many other coronaviruses. The researchers noted that the SARS-CoV-2 spike protein's receptor-binding domain (“RBD”) is optimized to bind the human cell receptor angiotensin-converting enzyme 2 (ACE2) and that each subunit of the spike protein trimer has a polybasic cleavage site. The researchers found that these cleavage sites are distributed about 10 nm away from the RBD, and can enhance the binding affinity between the SARS-CoV-2 and ACE2 thereby suggesting drug treatment therapies that utilize molecules that can neutralize these polybasic cleavage sites. See B. Qiao and M. Olvera de la Cruz, CS Nano 2020, 14, 8, 10616-10623, Publication Date: Aug. 2, 2020, https://doi. org/10.1021/acsnano.0c04798.

It is well understood that covalent and noncovalent bonds differ in their strength. Covalent bonds are the strongest and comprise a stable chemical force holding atoms in molecules together by sharing one or more pairs of electrons. Noncovalent interactions are somewhat weaker and involve the weak sharing of an electron pair between a hydrogen atom and another atom. Id. Van der Waals forces are involved which can cause inherent instability of non-covalent substances. A non-covalent interaction differs from a covalent bond in that it does not involve the sharing of electrons, but rather involves more dispersed variations of electromagnetic interactions between molecules or within a molecule. See Lodish H, Berk A, Zipursky S L, et al. Molecular Cell Biology. 4th edition. New York: W. H. Freeman; 2000. Glossary. Available from: https://www.ncbi.nlm.nih. gov/books/NBK21607/. See also Wikipedia contributors. (Jun. 29, 2021). Non-covalent interaction. In Wikipedia, The Free Encyclopedia. Retrieved 7/2/2021, from https://en. wikipedia.org/w/index.php?title=Non-covalent_interaction&oldid=1031050566.

Losasso et al. conducted studies resulting in findings that imply that the potency of antimicrobial peptides may not be a purely intrinsic chemical property and, instead, depends on the mechanical state of the target membrane. See Valeria Losasso, Ya-Wen Hsiao, Fausto Martelli, Martyn D, Winn, and Jason Crain, “Modulation of Antimicrobial Peptide Potency in Stressed Lipid Bilayers”. Phys. Rev. Lett. 122, 208103 (24 May 2019)(Abstract). See also Jason Cain, “Focusing on Cell Membranes to Fight Antibiotic Resistance”, IBM Research Blog, ibm.com/blogs/research/2019/05/cell-membranes-antibiotic-resistance/(24 May 2019)(commenting on the work of Losasso et al. and noting that the researchers discovered a relationship between membrane tension, generated by osmotic pressure or environmental forces, for example, and chemical interactions of antimicrobial agents, with both working to disrupt the bacterial membrane.)

Pandur et al studied liposome (lipid vesicle) destruction by hydrodynamic cavitation in comparison to chemical, physical and mechanical treatments. Liposomes are used extensively as model systems to study properties and stability of lipid bilayers to different physico-chemical or biochemical parameters. Pandur et al. concluded that hydrodynamic cavitation was among the most effective physico-chemical treatments in destroying lipid vesicles. See Pandur Z̆, Dogsa I, Dular M, Stopar D. Liposome destruction by hydrodynamic cavitation in comparison to chemical, physical and mechanical treatments. Ultrason Sonochem. 2020 March; 61: 104826. doi.: 10.1016/j.ultsonch.2019.104826. Epub 2019 Oct. 19. PMID: 31670247. Abu-Farha et al. report that SARS-COV-2 is an enveloped virus that is surrounded by a lipid bilayer. See Abu-Farha M, Thanaraj T A, Qaddourni M G, Hashern A, Abubaker J, Al-Mulla F. “The Role of Lipid Metabolism in COVID-19 Virus Infection and as a Drug Target” Int J. Mol Sci. 2020 May 17; 21(10):3544. doi: 10.3390/ijms21103544. PINED: 32429572; PMCID: PMC7278986. Abu-Farha et al. discuss the importance of lipid metabolism in the design of antiviral drugs, indicating that more work is needed to focus on the role played by lipid species at the structure and signaling pathway level in the viral life cycle. This will allow Abu-Farha et al. to establish new drug targets as well as enhance existing drugs. Such lipid-based therapies can be used alone or in combination with other drugs, but they will have the advantage of targeting multiple viruses.

Peng et al. (CN111358647(A), “Hyperbaric oxygen chamber for treatment of infectious diseases and use method of hyperbaric oxygen chamber” (7/3/2020 abstract) disclose a monoplace hyperbaric oxygen chamber for treatment of infectious diseases and a use method of the hyperbaric oxygen chamber, stated to be especially suitable for treatment of COVID-19. The hyperbaric oxygen chamber includes a chamber-purifying hospital infection-preventing system, a hyperbaric oxygen treatment system, an exhaust disinfection purification system and an exhaust recycling system. The chamber-purifying hospital infection-preventing system includes a chamber body, pressurized air inlets communicating with a compressed air source are arranged in two sides of the top of the chamber body, and air discharging openings are arranged in two sides of the bottom of the chamber body, the inner upper part of the chamber body is provided with a porous plate, and an air inlet buffer zone is formed between the pressurized air inlets and the porous plate, so that the compressed air introduced from the pressurized air inlets flows downwards from a vertical layer of the inner top of the chamber body. According to Peng et al., their hyperbaric oxygen chamber for treatment of the infectious diseases is beneficial to the hyperbaric oxygen treatment of the infectious diseases, and can not only prevent and control the spread of pathogens of the infectious diseases, but also improve the treatment effect on the infectious diseases.

Hadanny et al. have also suggested the use of HBOT for treatment of severe COVID-19 patients, showing in what is thought to be the first randomized controlled study of the short-term effects of HBOT in severe COVID-19 patients that HBOT is feasible, and can improve oxygenation and clinical status and decrease inflammation in patients with respiratory failure due to COVID-19. These authors studied the effects of HBOT in COVID-19 patients using a prospective randomized controlled design among 30 severely ill COVID-19 patients admitted between May 1, 2020 and Oct. 15, 2020 (clinical trials.gov, NCT04358926). The HBOT protocol was administrated in a Monoplace chamber BLKS-303 model (Khrunichev State Research and Production Space Center, Russia), located within the COVID-19 unit. The protocol comprised of eight consecutive sessions, two sessions per day, four days consecutively. Each session included breathing 100% oxygen at 2.2 absolute atmospheres (ATA) for 60 minutes with no air breaks. Compression/decompression rates were 1 meter/minute. All sessions were supervised by a hyperbaric medicine trained physician and hyperbaric trained nurse at all times. To reduce oxygen toxicity effects, the protocol used was composed of oxygen at 2.2 ATA for 60 minutes. See Hadanny, et al., Hyperbaric Oxygen Therapy for COVID-19 Patients: A Prospective, Randomized Controlled Trial. Available at SSRN: https://ssrn. com/abstract=3745115 or http://dx.doi.org/10.2139/ssrn.3745115.

Other researchers are evaluating the use of HBOT to treat COVID-19. See, e.g., Moon R E, Weaver L K. Hyperbaric oxygen as a treatment for COVID-19 infection? Undersea Hyperb Med. 2020 Second-Quarter; 47(2):177-179. PMID: 32574432 (Abstract)(noting that refractory hypoxemia is certainly treatable with HBOT due to the obvious effect of increasing inspired oxygen partial pressure (PO₂), the major reason for using HBO₂ for its established indications); Paganini M. et al. (Jul. 22, 2020) The Role of Hyperbaric Oxygen Treatment for COVID-19: A Review. In: Pokorski M. (eds) Medical and Biomedical Updates. Advances in Experimental Medicine and Biology, vol 1289. Springer, Cham. https://doi.org/10.1007/5584_2020_568 (Abstract) (indicating the large variability in protocols and exposure frequency, and summarizing biological mechanisms of action of increased O₂ pressure, hoping to clarify more appropriate protocols and more useful application of HBO in COVID-19 treatment); Maio A D, Hightower L E. COVID-19, Acute respiratory distress syndrome (ARDS), and hyperbaric oxygen therapy (HBOT): what is the link? Cell Stress Chaperones. 2020 May; 25:717-720. Published online 5/18/2020. https://doi.org/10.1007/s12192-020-01121-0, PMCID: PMC7232923 (commenting on the potential utility of HBOT to treat COVID-19 along with discussion of logistics issues for making HBOT available in an ICU); Kjellberg A, De Maio A, Lindholm P. Can hyperbaric oxygen safely serve as an anti-inflammatory treatment for COVID-19? Med Hypotheses. 2020 November; 144:110224. doi: 10.1016/j.mehy.2020.110224. Epub 2020 Aug. 30. PMID: 33254531; PMCID: PMC7456590 (noting that HBO₂ is used in clinical practice to treat inflammatory conditions but has not been scientifically evaluated for COVID-19. Experimental and empirical data suggests that HBO₂ may reduce inflammatory response in COVID-19 but concerns remain regarding pulmonary safety in patients with pre-existing viral pneumonitis.

Although vaccine development has begun to address the Covid-19 pandemic, there remains the reality that variants emerge, or the possibility that other pandemic-like infectious disease outbreaks occur, thereby continuing the need to develop effective treatment strategies independent of vaccine development. Furthermore, even with the current availability of vaccines for, e.g., COVID-19, medical researchers are discovering long-term health effects occurring in persons who were once infected with COVID-19. As studies on treatment of COVID-19 evolve, medical data is showing that some patients who survived a COVID-19 infection are experiencing what researchers refer to as “Long-haul COVID-19” or “post-COVID-19 syndrome”. Thus, the treatment of the COVID-19 virus must consider not only the active infection, but also the side effects of a patient resulting from such infection. For example, Hadanny et al. also propose a clinical trial (slated to have started on Jan. 1, 2021) to study the use of HBOT as a method for treatment of post-COVID-19 syndrome, which is characterized by cognitive impairment, sleep disorders, smell and taste disorders, pain and more. Hadanny et al., “Hyperbaric Oxygen Therapy for Post-COVID-19 Syndrome (HBOTpCOVID)”, HIH, clinicaltrials. gov/ct2/show/NCT04647656 (Dec. 1, 2020).

It has also been proposed hypothetically to use grounded aircraft to provide a treatment area within the pressurizable aircraft cabin, that in combination with oxygen therapy, can be pressurized to provide a treatment protocol much like with HBOT. See, D. Cooper, “The unlikely plan to save COVID-19 patients with planes—Grounded aircraft could make ad-hoc hyperbaric chambers for patients.” www.engadget.com/covid-19-hyperbaric-chamber-163005245.html, 4/27/2020, last accessed 6/25/2021.

These HBOT methodologies (whether performed in traditional hyperbaric chamber or in an ad-hoc pressurized airline cabin) are directed at making it easier for the patients to breath, particularly in those patients suffering from ARDS, but do not disclose or suggest the use of hyperbaric chambers to create pressure differentials across the virus (pathogen) outer membrane to destroy the pathogen or otherwise disable its functionality. Therefore, there remains a need for a pathogenic disease treatment regimen to be carried out in a closed environment like a hyperbaric chamber or even a pressurized airplane cabin using fluctuating temperature, humidity and/or pressure to weaken the outer membrane of the pathogen, e.g., to disrupt the spike glycoproteins on the surface of an individual coronavirus to weaken or disable the virus. Likewise, there remains a need for a pathogenic disease treatment regimen that employs vibrations and/or magnetic fluctuations from ultra-sound devices, MRIs and the like that can reach the active pathogen (e.g., coronavirus) and likewise weaken the outer membrane of the pathogen, e.g., to disrupt the spike glycoproteins on the surface of an individual coronavirus to weaken or disable the virus, such treatment regimen being enhanced when optionally carried out in a closed environment like a hyperbaric chamber or even a pressurized airplane cabin using a combination of fluctuating temperature, humidity and/or pressure.

Therefore, there continues to exist an urgent need for treatment methods that can be readily deployed to treat humans suffering from a pandemic viral infection, particularly COVID-19, including the long-term effects caused by the COVID-19 infection, such as post-COVID-19 syndrome or “long-haul COVID-19”. There also continues to be a need to provide effective, large scale HBOT treatment facilities for treatment of, e.g., other potential viral and bacterial infections, other infectious disease, various medical conditions, including age-related cognitive and functional decline, fibromyalgia, chronic wounds, post-concussion syndrome, Alzheimer's, inflammation, or other maladies where treatment can be enhanced via increasing the oxygenation within the patient, as well as for use for aesthetic therapies or treatments, etc.

Additionally, given the urgent need for medical staffing during a pandemic, resources at medical treatment centers have been strained, due to the unexpected high volumes of patients arriving requiring urgent care. As such, one of the first lines of protection for medical workers is to use medical cloth/PPE masks to cover their noses and mouths. However, when there is a shortage of masks, it may become necessary to reuse masks after they have been sterilized. Within the medical field, autoclaves are used for sterilization and typically employ a high-pressure steam-filled chamber (pressurized saturated steam at 250° F. for about 15-20 minutes). However, cloth/PPE medical face masks likely cannot survive the high temperatures, so combining heat and pressurized water likely is not feasible for sterilization of a mask potentially exposed to a novel virus. Furthermore, use of gas sterilization techniques can damage the efficiency of a medical face mask. See Garcia, Jennifer, “Face Mask Type Matters When Sterilizing, Study Finds”, Internal Medicine News, 6/18/2020. www.mdedge. com/internalmedicine/article/224116/coronavirus-updates/face-mask-type-matters-when-sterilizing-study, last accessed 6/27/2020. Anderegg et al, report the use of convection oven heat and humidity (85° C., 30 min., 60-85% humidity) for a scalable decontamination of N95 respirators in hospital settings during the COVID-19 crisis, but suggesting that further work was needed to ensure viral inactivation of SARS-CoV-2 on these Filtering Facepiece Respirators (FFRs). See Anderegg L, Meisenhelder C, Ngooi C O, Liao L. Xiao W, et al, (2020) “A scalable method of applying heat and humidity for decontamination of N95 respirators during the COVID-19 crisis.” PLOS ONE 15(7):e0234851. https://doi.org/10.1371/journal.pone.0234851. As such, there also remains a need to enhance the ability to sterilize surgical masks without significant drop in efficiency to permit ready reuse.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

There is disclosed a pathogen treatment regimen carried out in a closed environment like a hyperbaric chamber or even a pressurized airplane cabin, where combinations of fluctuating temperature, humidity and/or pressure are employed to weaken the pathogen, e.g., to disrupt the spike glycoproteins that surround each individual coronavirus, to weaken or disable the virus. This method could also be practiced along with HBOT practices. There is also disclosed the use of vibrations and/or magnetic fluctuations from ultra-sound devices, ultrasonic cavitation devices, vibration devices, acoustic devices, MRIs, magnetic field generators and/or the like that can reach internally into the subject patient to the pathogen (e.g., virus) and likewise weaken the pathogen, e.g., to disrupt the spike glycoproteins that surround each individual coronavirus, to weaken or disable the virus. There is also disclosed a pathogen treatment regimen carried out in a closed environment like a hyperbaric chamber or even a pressurized airplane cabin, where combinations of fluctuating temperature, humidity and/or pressure are employed in combination with application of vibrations and/or magnetic fluctuations from ultra-sound devices, ultrasonic cavitation devices, vibration devices, acoustic devices, MRIs, magnetic field generators and/or the like that can reach internally into the subject patient to the pathogen (e.g., virus) and likewise weaken the pathogen, e.g., to disrupt the spike glycoproteins that surround each individual coronavirus, to weaken or disable the virus. These methods are directed to treating subject patients selected from the group consisting of humans, animals, plants, and insects, including livestock, cattle, sheep, goats, equines, dogs, cats, birds, poultry, reptiles, food crops, and beneficial insects such as bees and earthworms.

In one embodiment of the present disclosure there is disclosed a method of treating a subject patient afflicted with a pathogenic disease caused by a pathogen, the pathogen comprising an outer membrane structure maintaining an internal pressure inside of the membrane. This method could be used, e.g., to treat a human infected with a virus, such as the novel coronavirus pathogen and its variants, the human being comprising a respiratory system with lungs, the virus comprising an outer membrane maintaining an internal pressure inside of the membrane. In this embodiment, the method comprises the steps of: (1) placing the patient in a hyperbaric chamber capable of being pressurized or depressurized to a desired internal chamber pressure; (2) administering oxygen in the chamber to a desired level to enhance oxygenation levels in the human being's body (e.g., lungs); and (3) adjusting the internal chamber pressure sufficient to cause a pressure differential between the pathogen (e.g., virus) internal pressure and the internal chamber pressure to cause the outer membrane of the pathogen (e.g., virus) to rupture or to otherwise disable the pathogen (e.g., virus).

The pathogen may be a virus, a bacterium, a fungus, a parasite, worm, protozoa, or helminth. The viral infection could be any viral infection, including infection by a novel coronavirus and its variants. In one embodiment, the novel coronavirus is SARS-CoV-2 and its variants, now known and later discovered, and the human being is suffering from COVID-19 or post-COVID-19 syndrome. The method step of adjusting the internal chamber pressure can be achieved by increasing the internal chamber pressure. The pressure can be increased between about 1.0 atm up to about 7 atm according to the tolerances of the human being infected with the pathogen. In another embodiment the step of adjusting the internal chamber pressure is achieved by decreasing the internal chamber pressure, e.g., to about 0.5 atm or less according to the tolerances of the human being infected with the pathogen (e.g., coronavirus).

In these embodiments, a series of pressure adjustments may be employed, for example, the series of pressure adjustments may comprise incremental step pressure increases, incremental step pressure decreases and/or cycling between incremental step pressure increases and incremental step pressure decreases. Likewise, in these embodiments, a series of oxygen level adjustments may be employed, for example, the series of oxygen level adjustments may comprise incremental step oxygen level increases, incremental step oxygen level decreases and/or cycling between incremental step oxygen level increases and incremental step oxygen level decreases.

The hyperbaric chamber may be a single occupant or multi-occupant unit. The hyperbaric chamber may also comprise a pressurized full body suit worn by the human being. The hyperbaric chamber may also comprise an aircraft cabin capable of being pressurized. These pressure-differential methods may further comprise the step of administering breathing assistance to the human. Additionally, the method may be enhanced by performing ultrasonic cavitation or ultrasound therapy and/or magnetic field therapy on an exterior torso region of the human being proximate the human being's lungs. Also, these methods may be further enhanced by comprising one or more of the following steps: (1) varying the temperature in the chamber; (2) varying the humidity in the chamber; (3) varying the oxygen level received by the patient; and/or (4) infusing additional gases into the chamber (or directly into the lungs of the human being) the additional gasses selected from the group consisting of: known respiratory inhaler gases such as those used in the treatment of asthma and COPD, e.g., corticosteroid inhalers (such as fluticasone), and bronchodilator inhalers (such as albuterol); inhalers infused with nasal decongestants (including natural menthol and camphor based products); natural inhalable substances, e.g., herbal inhalers, aromatherapeutics; medical grade oxygen, medical grade ethyl alcohol vapor or nebulized mist, and treatment gases used for clearing mucus plugs in the human being's lungs.

Also described is a mobile method of treating one or more human patients afflicted with a pathogenic disease, such as the COVID-19 viral infection, the pathogen (e.g., virus, bacteria, fungi, parasite, protozoa, worm, helminth) comprising an outer membrane maintaining an internal pressure inside of the membrane, the mobile method comprising the steps of: (1) placing the one or more human patients in an air transport vehicle located on the ground, the air transport vehicle capable of obtaining in-flight altitudes where the external air pressure ranges between about 0.3-0.2 atm, the air transport vehicle further comprising: (i) a pressurizable fuselage integral to the air transport vehicle, the fuselage having climate control and oxygen supply; (ii) one or more independently pressurizable treatment chambers located within the fuselage of the air transport vehicle, the one or more pressurizable treatment chambers further comprising climate control independent of the fuselage, pressure control independent of the fuselage, oxygen supply independent of the fuselage, one or more seats, beds or treatment tables for receiving and securing the one or more human patients, and medical supplies for maintaining the life of the one or more human patients; (iii) controllers for adjusting the climate in the one or more pressurizable treatment chambers, including for regulation of temperature, humidity and oxygen levels; and (iv) controllers for adjusting the pressure in the one or more pressurizable treatment chambers; (2) flying the air transport vehicle to an altitude having a desired ambient external air pressure; (3) independently adjusting the air pressure in the one or more independently pressurizable treatment chambers to a desired treatment pressure sufficient to cause a pressure differential between the pathogen (e.g., virus) internal pressure and the internal chamber pressure to cause the outer membrane of the pathogen (e.g., virus) to rupture or to otherwise disable the pathogen (e.g., virus); (4) maintaining the desired treatment pressure for a desired length of time; and (5) returning the air transport vehicle to the ground.

This mobile method may further comprise the steps of varying the pressure in the one or more treatment chambers while the air transport vehicle is in flight to induce differential pressures sufficient to rupture or disrupt the pathogen (e.g., virus) membrane and/or administering breathing assistance to the one or more human patients. In this mobile method, the air pressure in the one or more independently pressurizable treatment chambers may be independently adjusted upwardly or downwardly. The pressures may be increased up to about 7 atm or to an upper limit value permitted in the air transport vehicle or the chambers therein. The pressures may be decreased to about 0.5 atm or to a lower limit value permitted in the air transport vehicle, or chambers therein, or as can be tolerated by the one or more human patients. A series of pressure adjustments may be employed, such as, for example, incremental step pressure increases, incremental step pressure decreases, and/or cycling between incremental step pressure increases and incremental step pressure decreases. Likewise, in these embodiments, a series of oxygen level adjustments may be employed, for example, the series of oxygen level adjustments may comprise incremental step oxygen level increases, incremental step oxygen level decreases and/or cycling between incremental step oxygen level increases and incremental step oxygen level decreases.

There is further disclosed a method of treating one or more human patients afflicted with a pathogenic disease caused by a pathogen, such as for example, a COVID-19 viral infection, the pathogen comprising an outer membrane maintaining an internal pressure inside of the membrane, the method comprising the steps of: (1) placing the one or more human patients in an air transport vehicle located on the ground; (2) pressurizing the one or more treatment chambers to a desired pressure sufficient to cause a pressure differential between the pathogen internal pressure and the internal chamber pressure to cause the outer membrane of the pathogen to rupture or to otherwise disable the pathogen; and (3) returning the pressure in the one or more treatment chambers to an ambient pressure. In this embodiment, the air transport vehicle further comprises (i) a pressurizable fuselage integral to the air transport vehicle, the fuselage having climate control and oxygen supply; (ii) one or more a pressurizable treatment chambers located within the fuselage of the air transport vehicle, the one or more pressurizable treatment chamber further comprising, climate control independent of the fuselage, pressure control independent of the fuselage, oxygen supply independent of the fuselage, one or more seat/bed/table for securing one or more human patients, medical supplies for maintaining the life of the one or more human patients; (iii) controllers for adjusting the climate in the one or more pressurizable treatment chambers; and (iv) controllers for adjusting the climate in the one or more pressurizable treatment chambers. In one embodiment, the treatment chambers are pressurized by the pressurizable fuselage. In one embodiment, the treatment chambers are the existing seats in the air transport vehicle cabin.

There is also disclosed a mobile treatment unit for treating one or more human patients afflicted with a pathogenic disease caused by a pathogen such as for example the virus strains and variants causing COVID-19, the pathogen comprising an outer membrane maintaining an internal pressure inside of the membrane, the mobile treatment unit comprising: (1) an air transport vehicle capable of flying to altitudes where the external air pressure ranges between about 0.3-0.2 atm; (2) a pressurizable fuselage integral to the air transport vehicle, the fuselage having climate control and oxygen supply; (3) one or more independently pressurizable treatment chambers located within the fuselage of the air transport vehicle, the one or more pressurizable treatment chamber further comprising (i) climate control independent of the fuselage, (ii) pressure control independent of the fuselage, (iii) oxygen supply independent of the fuselage, (iv) one or more seats, beds or treatment tables for receiving and securing the one or more human patients, and (v) medical supplies for maintaining the life of the one or more human patients; (4) controllers for adjusting the climate in the one or more pressurizable treatment chambers; and (4) controllers for adjusting the pressure in the one or more pressurizable treatment chambers.

The use of these treatment methodologies has application to the treatment of pathogenically infected subject patients as well as to the treatment of subject patients in post-infection recovery, e.g., to treat or help prevent post-COVID-19 syndrome or other adverse side effects resulting from the pathogenic disease. Furthermore, the use of these methodologies have application in other areas of medicine, such as for example, treating wounds, the effects of aging, inflammation, and the effects of other maladies.

Also disclosed herein is a nonsurgical method of destroying a pathogen present in a subject patient, such as a viral pathogen causing COVID-19 located in a human subject patient's respiratory system, the human subject patient comprising, e.g., an upper torso and lungs laying within the upper torso, the method comprising the steps of identifying a body region of the subject patient afflicted with the pathogen; and performing an ultrasonic cavitation, ultrasound procedure or vibrational force therapy on an outer surface of the afflicted body region of the subject patient. If desired, this method can be modified by including the following additional steps: (1) before the step of performing the ultrasonic cavitation, placing the subject patient in a hyperbaric chamber capable of being pressurized or depressurized to a desired internal chamber pressure; and (2) adjusting the internal chamber pressure to the desired internal chamber pressure. The method may also include adjusting the environment of the subject patient, such as the temperature and humidity of the hyperbaric chamber, and the levels of oxygen administered to the subject patient during the treatment, e.g., 100% oxygen, 100% oxygen interrupted with intervals of lower oxygen level, such as ambient air. HBOT techniques, and, if desired, the administering of additional treatment gases into the patient's lungs. Additionally, the temperature of the air or oxygen delivered to the subject patient could be varied. Additionally, the ultrasound procedure could be monitored using a combined ultrasound and MR imaging procedure to assist in guiding a focused ultrasound therapy. This ultrasound technique will have application to other parts of the body where the infection may reside. This method may also be employed to provide therapy to a patient suffering from post-infection complications such as post-COVID-19 syndrome.

Similarly, there is disclosed a nonsurgical method of destroying pathogens present in an anatomical location of a subject patient, the method comprising the steps of: identifying the anatomical location of the patient where the pathogen is present; and performing an ultrasonic procedure on a region of an outer surface of the subject patient proximate the identified anatomical location to direct ultrasonic forces toward the pathogen, e.g., a virus causing COVID-19 in such anatomical location. This method may further comprise the step of using a guided ultrasound procedure in tandem with Magnetic Resonance Imaging, and may also be carried out within a hyperbaric chamber while also providing hyperbaric oxygen treatment to the patient. This method may also be employed to provide therapy to a patient suffering from post-infection complications such as post-COVID-19 syndrome.

There is also disclosed a nonsurgical method of destroying pathogens present in an anatomical location of a subject patient, the method comprising the steps of: identifying the anatomical location of the patient where the pathogens are present; and applying a magnetic field force on a region of an outer surface of the subject patient proximate the identified anatomical location to direct the magnetic field forces toward the pathogen in such anatomical location. The magnetic field force may be selected from the group consisting of Magnetic Resonance Imaging (MRI) magnetic fields, pulsed magnetic fields, rotating magnetic fields, alternating magnetic fields, oscillating magnetic fields, magnetic nanoparticles, and magnetic hyperthermia.

There is also disclosed a method of treating a living subject or subject patient infected with a virus or other pathogen by the administration of magnetic forces, such as those provided by Magnetic Resonance Imaging (“MRI”) magnetic fields, pulsed magnetic fields, rotating magnetic fields, alternating magnetic fields, oscillating magnetic fields, dynamic magnetic fields, magnetic nanoparticles, and magnetic hyperthermia. This method could be used in combination with other of the methods disclosed herein, including ultrasound, ultrasonic cavitation, HBOT and/or the use of a hyperbaric chamber.

The above methods directed to treating humans afflicted with pathogenic disease could likewise be employed to treat other subject patients or living subjects, such as animals, plants, bee hives, and the like suffering from a pathogenic disease.

There is further disclosed a nonsurgical method of destroying fat cells in a human comprising the steps of: (1) placing the patient in a hyperbaric chamber capable of being pressurized or depressurized to a desired internal chamber pressure; (2) adjusting the internal chamber pressure to the desired internal chamber pressure; and (3) performing an ultrasonic cavitation procedure on a desired portion of an outer surface of the human where the fat cell destruction is desired. This method may be augmented by the additional steps of simultaneously providing HBOT therapies to the patient.

Similarly, an enhanced method of fatty tissue removal in a human being is disclosed, the fatty tissue comprising one or more adipose cells each having a generally spherical membrane surrounding a fat reservoir, the fat reservoir having an internal fat reservoir pressure, the method comprising the steps of: (1) placing the patient in a hyperbaric chamber capable of being pressurized or depressurized to a desired internal chamber pressure; (2) adjusting the internal chamber pressure sufficient to cause a pressure differential between the fat reservoir internal pressure and the internal chamber pressure to place stress on the one or more adipose cell generally spherical membranes; and (3) performing an ultrasonic cavitation procedure on a desired portion of an outer surface of the human where the fat cell destruction is desired. The method may also include adjusting the environment of the patient, such as the temperature and humidity of the hyperbaric chamber, and the levels of oxygen administered to the patient during the treatment, e.g., 100% oxygen, 100% oxygen interrupted with intervals of lower oxygen level, such as ambient air. It is likewise envisioned that these and other “spa-type” therapeutic or aesthetic treatments benefitting from the use of HBOT could be administered to large groups of patients on an aircraft cabin-based hyperbaric chamber located on the ground or during long flights.

The above-disclosed methods of fatty tissue removal or destruction likewise have application to animals, particularly to pets that require weight loss for medical health reasons.

There is also disclosed a method of disinfecting medical equipment contaminated with a pathogen, such as the viruses causing COVID-19, the pathogen comprising an outer membrane maintaining an internal pressure inside of the membrane, the method comprising the steps of: (1) placing the equipment in a hyperbaric chamber capable of being pressurized or depressurized to a desired internal chamber pressure; and (2) adjusting the internal chamber pressure sufficient to cause a pressure differential between the pathogen internal pressure and the internal chamber pressure to cause the outer membrane of the pathogen to rupture or to otherwise disable the pathogen. In this method, the desired internal chamber pressure may range between 0.0 atm to 100 atm, excluding 1 atm. The step of adjusting the internal chamber pressure may be carried out by rapidly increasing or decreasing the internal chamber pressure. Also, the step of adjusting the internal chamber pressure is carried out by cycling between increasing and decreasing the internal chamber pressure. In one embodiment, the medical equipment comprises surgical masks.

It is noted that in this disclosure and particularly in the claims, terms such as “comprises”, “comprised”, “comprising” and the likes can have the meaning attributed to them in U.S. Patent law; e.g., they can mean “includes”, “included”, “including”, and the likes; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

Certain embodiments will hereafter be described with reference to the accompanying drawings, wherein like reference numerals denote like elements. It should be understood, however, that the accompanying drawings illustrate only the various implementations described herein and are not meant to limit the scope of various technologies described herein. The drawings are as follows:

FIG. 1A is a schematic representation of a virus, such as the novel coronavirus.

FIG. 1B is a schematic representation of soap molecules from a bar of soap.

FIG. 1C represents the interaction of the soap molecules of FIG. 1B with the virus of FIG. 1A.

FIG. 2 is representative depiction of a virus, such as the coronavirus wherein the internal pressure of the virus is at equilibrium with the pressure external to the virus.

FIG. 3 is representative depiction of a virus, such as the novel coronavirus, being exposed to an external pressure greater than the internal pressure of the virus, thereby causing a pressure differential sufficient to cause the destruction of the virus (or its protective outer layer), to disable the virus, or to otherwise interfere with the virus functionality.

FIG. 4 is representative depiction of a virus, such as the novel coronavirus, being exposed to an external pressure that is less than the internal pressure of the virus, thereby causing a pressure differential sufficient to cause the destruction of the virus (or its protective outer layer), to disable the virus, or to otherwise interfere with the virus functionality.

FIG. 5 is a schematic representation of one embodiment of the present disclosure utilizing a hyperbaric chamber to apply the desired external pressure to a human patient infected with a virus such as the novel coronavirus.

FIG. 6 is a schematic representation of one treatment method of the present disclosure utilizing a hyperbaric chamber to apply a desired external pressure to a human patient infected with a virus such as the novel coronavirus. In this embodiment, the virus is thereby exposed to an external pressure greater than the internal pressure of the virus, thereby causing a pressure differential sufficient to cause the destruction of the virus (or its protective outer layer), to disable the virus, or to otherwise interfere with the virus functionality.

FIG. 7 is a schematic representation of another treatment method of the present disclosure utilizing a hyperbaric chamber to apply a desired external pressure to a human patient infected with a virus such as the novel coronavirus. In this embodiment, the virus is thereby exposed to an external pressure lesser than the internal pressure of the virus, thereby causing a pressure differential sufficient to cause the destruction of the virus (or its protective outer layer), to disable the virus, or to otherwise interfere with the virus functionality.

FIG. 8 is a schematic representation of an aircraft being used to treat virus-infected patients by using the cabin pressure of the aircraft to create the desired external pressure to be applied to the patient. In one embodiment, an external pressure greater than the virus internal pressure can be administered to the patients on board the aircraft while the aircraft remains on the ground (via cabin pressurization) or via adjustment of the cabin pressure while in flight at a desired altitude. The desired external pressure can be applied in varying protocols, such as, steady pressure increases, pulsed pressure increases, or variations between pressure increases and pressure decreases, and the like.

FIG. 9 is a schematic representation of an aircraft being used to treat virus-infected patients by using the cabin pressure of the aircraft to create the desired external pressure to be applied to the patient. In another embodiment, an external pressure lesser than the virus internal pressure can be administered to the patients on board the aircraft while the aircraft remains on the ground (via cabin pressurization and depressurization) or via adjustment of the cabin pressure while in flight at a desired altitude. The desired external pressure can be applied in varying protocols, such as, steady pressure increases, pulsed pressure increases, or variations between pressure increases and pressure decreases, and the like.

FIG. 10A is a schematic depiction of a set of coronaviruses penetrating adipose tissue from a sample collected from a patient. This depiction is based generally on a photo having the following photo credit: Niaid/Planet Pix via Zuma Press/Cordon Press, in “Real photographs of the coronavirus under the microscope”, National Geographic, www. nationalgeographic.com.es/ciencia/fotografias-reales-coronavirus-bajo-microscopio_15335/1, last accessed 7/5/2020.

FIG. 10B is an illustration modifying the depiction of FIG. 10A to schematically hypothetically depict what the infected adipose tissue should look like after the proposed pressure treatments described herein have destroyed the outer coating or membrane of the coronavirus.

FIG. 11 is a schematic representation of another treatment method of the present disclosure utilizing a hyperbaric chamber to apply a desired external pressure to personal protective equipment (PPE), such as, masks, gloves, goggles, jumpsuits, or other PPE in need of sterilization against potential contamination from a virus, such as coronavirus. In this embodiment, the virus is thereby exposed to an external pressure greater than the internal pressure of the virus, thereby causing a pressure differential sufficient to cause the destruction of the virus (or its protective outer layer), to disable the virus, or to otherwise interfere with the virus functionality. In another embodiment, the virus is thereby exposed to an external pressure lesser than the internal pressure of the virus, thereby causing a pressure differential sufficient to cause the destruction of the virus (or its protective outer layer), to disable the virus, or to otherwise interfere with the virus functionality.

FIG. 12 is a schematic representation of an ultrasonic cavitation procedure being performed in a hyperbaric chamber to apply a desired external pressure to a human patient undergoing such procedure according to another embodiment of the present disclosure.

FIG. 13A is a schematic representation of an ultrasonic cavitation procedure being applied to a human patient's external torso in the region of the lungs to assist in destruction or disabling of a virus such as the coronavirus infecting the patient's lungs according to another embodiment of the present disclosure.

FIG. 13B is a schematic representation of an ultrasonic cavitation procedure being applied to a human patient's external torso in the region of the lungs to assist in destruction or disabling of a virus such as the coronavirus infecting the patient's lungs according to another embodiment of the present disclosure. In this embodiment, this cavitation procedure is performed within a hyperbaric chamber under increased or decreased external pressure as described in connection with FIGS. 6 and 7.

FIG. 14A is a schematic representation of an aircraft embodiment stationed on the ground wherein the cabin will be used as a hyperbaric chamber according the disclosures herein. In this embodiment, the aircraft utilizes its own engine power to, e.g., pressurize the cabin and provide power, but is provided with an external source of medical grade oxygen for use within the cabin, or via masks worn by patients on the aircraft. In this embodiment, the external source of oxygen is provided by a tank of pressurized oxygen (gas or liquid state) located proximate the aircraft.

FIG. 14B is a schematic representation of an aircraft embodiment stationed on the ground wherein the cabin will be used as a hyperbaric chamber according the disclosures herein. In this embodiment, the aircraft utilizes its own engine power to, e.g., pressurize the cabin and provide power, but is provided with an external source of medical grade oxygen for use within the cabin, or via masks worn by patients on the aircraft. In this embodiment, the external source of oxygen is provided by a tank of pressurized oxygen (gas or liquid state) mounted on a mobile vehicle that can be positioned proximate the aircraft.

FIG. 14C is a schematic representation of an aircraft embodiment stationed on the ground wherein the cabin will be used as a hyperbaric chamber according the disclosures herein. In this embodiment, the aircraft utilizes its own engine power to, e.g., pressurize the cabin and provide power, but is provided with an external oxygen generator or concentrator to provide medical grade oxygen for use within the cabin, or via masks worn by patients on the aircraft. In this embodiment, the external source of oxygen is provided by a fixed oxygen generator/concentrator located proximate the aircraft.

FIG. 14D is a schematic representation of an aircraft embodiment stationed on the ground wherein the cabin will be used as a hyperbaric chamber according the disclosures herein. In this embodiment, the aircraft utilizes its own engine power to, e.g., pressurize the cabin and provide power, but is provided with an external oxygen generator or concentrator to provide medical grade oxygen for use within the cabin, or via masks worn by patients on the aircraft. In this embodiment, the external source of oxygen is provided by an oxygen generator/concentrator located on a mobile vehicle that can be positioned proximate the aircraft.

FIG. 14E is a schematic representation of an aircraft embodiment stationed on the ground wherein the cabin will be used as a hyperbaric chamber according the disclosures herein. In this embodiment, the aircraft utilizes its own engine power to, e.g., pressurize the cabin and provide power, but is provided with an oxygen generator or concentrator mounted within the aircraft, e.g., in the cargo hold, to provide medical grade oxygen for use within the cabin, or via masks worn by patients on the aircraft.

FIG. 15A is a schematic representation of an aircraft embodiment stationed on the ground wherein the cabin will be used as a hyperbaric chamber according the disclosures herein. In this embodiment, the aircraft does not utilize its engines, but instead, utilizes an external source of power to, e.g., pressurize the cabin and provide power, and an external source of medical grade oxygen for use within the cabin, or via masks worn by patients on the aircraft. In this embodiment, the external source of oxygen is provided by a tank of pressurized oxygen (gas or liquid state) located proximate the aircraft, and the power generator is in a fixed location proximate the aircraft.

FIG. 15B is a schematic representation of an aircraft embodiment stationed on the ground wherein the cabin will be used as a hyperbaric chamber according the disclosures herein. In this embodiment, the aircraft does not utilize its engines, but instead, utilizes an external source of power to, e.g., pressurize the cabin and provide power, and an external source of medical grade oxygen for use within the cabin, or via masks worn by patients on the aircraft. In this embodiment, the external source of oxygen is provided by a tank of pressurized oxygen (gas or liquid state) located on a mobile vehicle capable of being positioned proximate the aircraft, and the power generator is located on a mobile vehicle capable of being positioned proximate the aircraft.

FIG. 16A is a schematic representation of an aircraft embodiment stationed on the ground wherein the cabin will be used as a hyperbaric chamber according the disclosures herein. In this embodiment, the aircraft does not utilize its engines, but instead, utilizes an external source of power, an external pressure generator, and an external source of medical grade oxygen for use within the cabin, or via masks worn by patients on the aircraft. In this embodiment, the external source of oxygen is provided by a tank of pressurized oxygen (gas or liquid state) located proximate the aircraft, and the power generator and pressure generator are in fixed locations proximate the aircraft.

FIG. 16B is a schematic representation of an aircraft embodiment stationed on the ground wherein the cabin will be used as a hyperbaric chamber according the disclosures herein. In this embodiment, the aircraft does not utilize its engines, but instead, utilizes an external source of power, an external pressure generator, and an external source of medical grade oxygen for use within the cabin, or via masks worn by patients on the aircraft. In this embodiment, the external source of oxygen is provided by a tank of pressurized oxygen (gas or liquid state) located on a mobile vehicle positionable proximate the aircraft, and the power generator and pressure generator are in located on one or more mobile vehicles positionable proximate the aircraft.

FIG. 17A is a schematic representation of an aircraft cabin interior where patients are receiving hyperbaric oxygen treatments while the aircraft is stationed on the ground wherein the cabin will be used as a hyperbaric chamber according the disclosures herein and the patients will be administered oxygen via masks as desired.

FIG. 17B is a schematic representation of an aircraft cabin interior where patients are receiving hyperbaric oxygen treatments while the aircraft is stationed on the ground wherein the cabin will be used as a hyperbaric chamber according the disclosures herein and the patients will be administered oxygen via masks as desired, here via the use of portable oxygen generators.

FIG. 18A is a schematic representation of one or more stationary aircraft fuselages (without aircraft's wings or engines) wherein the cabin will be used as a hyperbaric chamber according the disclosures herein. In this embodiment, the aircraft does not utilize its engines, but instead, utilizes an external source of power, an external pressure generator, and an external source of medical grade oxygen for use within the cabin, or via masks worn by patients on the aircraft. In this embodiment, the external source of oxygen is provided by a tank of pressurized oxygen (gas or liquid state) located proximate the fuselage, and the power generator and pressure generator are in fixed locations proximate the fuselage.

FIG. 18B is a schematic representation of one or more stationary aircraft fuselages (without aircraft's wings or engines) wherein the cabin will be used as a hyperbaric chamber according the disclosures herein. In this embodiment, the aircraft does not utilize its engines, but instead, utilizes an external source of power, an external pressure generator, and an external source of medical grade oxygen for use within the cabin, or via masks worn by patients on the aircraft. In this embodiment, the external source of oxygen is provided by a tank of pressurized oxygen (gas or liquid state) located on a mobile vehicle positionable proximate the fuselage, and the power generator and pressure generator are located on one or more mobile vehicles positionable proximate the fuselage.

FIG. 18C is another schematic representation of one or more stationary aircraft fuselages (without aircraft's wings or engines) wherein the cabin will be used as a hyperbaric chamber according the disclosures herein. In this embodiment, the aircraft does not utilize its engines, but instead, utilizes an external source of power, an external pressure generator, and an external source of medical grade oxygen for use within the cabin, or via masks worn by patients on the aircraft. In this embodiment, the external source of oxygen is provided by a tank of pressurized oxygen (gas or liquid state) located proximate the fuselage, and the power generator and pressure generator are in fixed locations proximate the fuselage.

### DETAILED DESCRIPTION

Reference throughout the specification to “one embodiment,” “an embodiment,” “some embodiments,” “one aspect,” “an aspect,” or “some aspects” means that a particular feature, structure, method, or characteristic described in connection with the embodiment or aspect is included in at least one embodiment of the present disclosure. Thus, the appearance of the phrases “in one embodiment” or “in an embodiment” or “in some embodiments” in various places throughout the specification are not necessarily all referring to the same embodiment. Furthermore, the particular features, structures, methods, or characteristics may be combined in any suitable manner in one or more embodiments. The words “including” and “having” shall have the same meaning as the word “comprising.” Moreover, inventive aspects lie in less than all features of a single disclosed embodiment. Thus, the claims following the Detailed Description are hereby expressly incorporated into this Detailed Description, with each claim standing on its own as a separate embodiment. Reference is now made to the drawings which depict preferred embodiments, but are not drawn to scale.

In various embodiments, the present disclosure is directed generally to treatment of humans (or animals, including pets and livestock, beneficial insects including pollinator bees, plant life and foodstock) infected with viruses, or afflicted with other infectious or pathogenic diseases. By way of exemplary teaching, the present disclosure is directed primarily to methods of destroying the coronavirus (or other pathogen) through pressure variations, or as described below, other mechanisms for destroying the outer membrane of the pathogen or otherwise interfering with its functionality, such as via application of ultrasonic and/or magnetic forces or combinations of ultrasonic and/or magnetic forces with the use of a hyperbaric chamber.

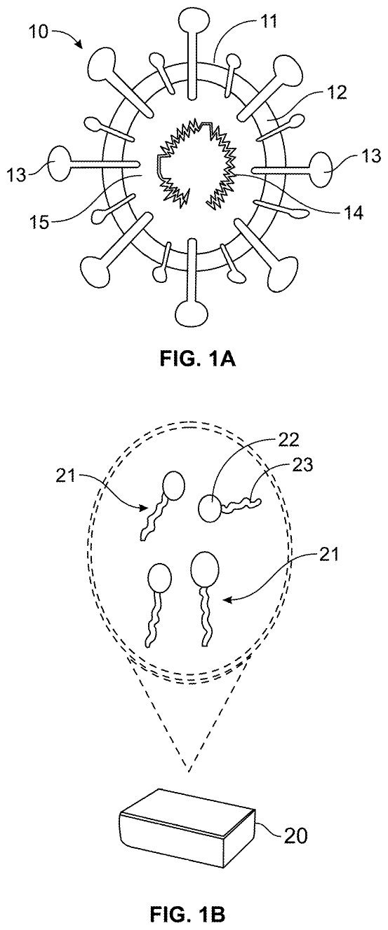

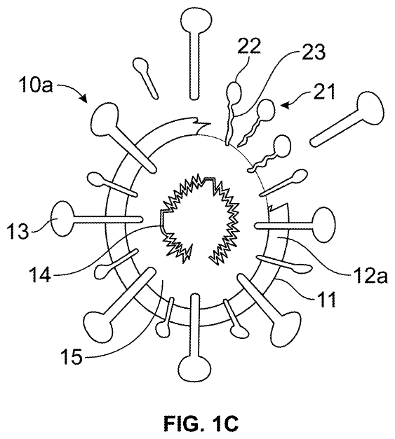

Referring to FIG. 1A, there is schematically shown a depiction of a virus such as a coronavirus 10. The coronavirus 10 is surrounded by a greasy bubble (outer membrane layer or envelop 12) understood to be a lipid bilayer protecting the genetic material 14 therein. The coronavirus 10 is characterized in part as having spike glycoproteins 13 extending outwardly from its surface 11. As noted, these spikes 13 are thought to be critical to the virus' ability to infect a human cell. The genetic material (RNA) 14 of the virus 10 is contained within the interior space 15 defined by the membrane 12. Referring also to FIG. 1B, there is depicted a source of soap 20 and a plurality of soap molecules 22 each having a hydrophilic head 22 that is attracted to water and a hydrophobic tail 23 that is attracted to grease, oil and lipid substances. Referring to FIG. 1C, it is known that soap can break down this lipid bilayer outer membrane bubble 12 destroying the functionality of the coronavirus 10 wherein the hydrophobic tail 23 interacts with and disrupts the membrane 12. The virus proteins and fragments are then washed away by the water. The SARS-CoV-2 virus is a self-assembled nanoparticle in which the weakest link is the lipid (fatty) bilayer 12. Soap dissolves the fat membrane 12 and the virus falls apart becoming an inactive virus 10a. The present disclosure presents methods of breaking this greasy bubble or lipid (fatty) bilayer 12 that surrounds the coronavirus 10 but in a different way, through pressure variations and physical disruptions across the bilayer 12.

The basic recommendation of the World Health Organization is to wash your hands with soap because soap breaks the greasy bubble (lipid bilayer 12) that surrounds the coronavirus. The present disclosure provides a method for breaking the greasy bubble 12 that surrounds the coronavirus by applying pressure variations to create pressure differentials, applying ultrasonic forces and/or applying magnetic forces that disrupt the virus (e.g. destroy or interfere with its membrane 12). Likewise, the present disclosure is directed to disabling the functionality of pathogens via the use of pressure differentials, ultrasonic and/or magnetic forces or combinations thereof.

Microorganisms and infectious agents such as viruses and virions and other pathogens play a major role in disease manifestations. The term “virus” is a broad, general term for any aspect of the infectious agent which can act as an obligate intracellular parasite, whereas a virion is an infectious particle in the extracellular phase of the host. Virus is a non-cellular, obligate parasite that is self-replicative inside a specific host cell. Virion is another form of a virus. The main difference between virus and virion is that virus is the nucleoprotein particle whereas virion is the active, infectious form of the virus. This present disclosure is directed to the destruction or disablement of, e.g., viruses, virions and virons. All viruses contain nucleic acid, either DNA or RNA (but not both), and a protein coat, which encases the nucleic acid. Some viruses are also enclosed by an envelope of fat and protein molecules. In its infective form, outside the cell, a virus particle is called a virion.

Coronavirus, is defined as any virus belonging to the family Coronaviridae. Coronaviruses have enveloped virions (virus particles) that measure approximately 120 nm (1 nm=10⁻⁹ metre) in diameter. Club-shaped glycoprotein spikes 13 in the envelope 12 give the viruses a crownlike, or coronal, appearance.

Specific reference herein to treatment methods directed to destroying or disabling a virus is a reference to any part of the virus, virion, viron, etc. capable of being destroyed or disabled as a means of treating or abating, e.g., a viral infection in, e.g., a person.

Referring to FIGS. 1A-1C there is illustrated generally how a virus 10 (or viron), such as the novel coronavirus causing the COVID-19 pandemic viral infection in humans, can be disrupted via the interaction of soap 20 with the external membrane/envelope 12 (greasy bubble) surrounding the virus. As depicted and presently understood, the virus, here for example the novel coronavirus 10 causing COVID-19, has its genetic material 14 residing in an interior space 15 within a lipid bylayer outer membrane 12. The coronavirus 10 also comprises spike glycoproteins 13 protruding outwardly from the outer surface 11 of the membrane 12. The hydrophobic tails 23 of the soap molecules 21 are attracted to and disrupt the lipid bilayer 12 of the coronavirus 10 creating a ruptured membrane 12a that renders the coronavirus 10a noninfectious.

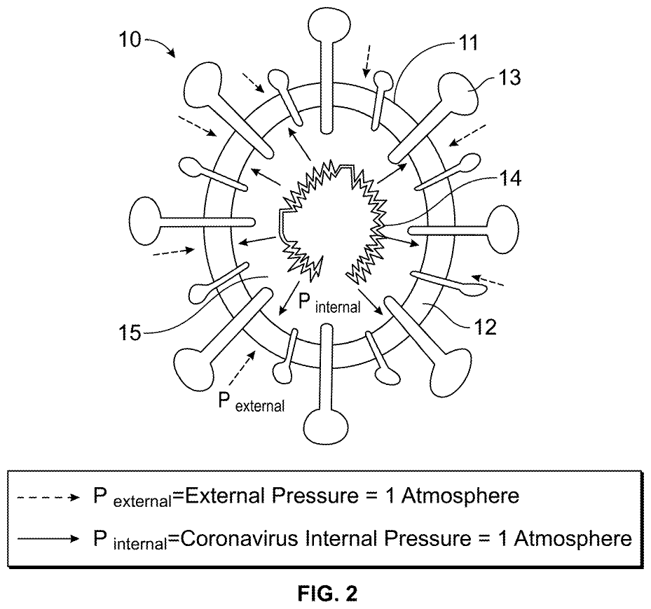

Referring also to FIG. 2, there is shown a representative depiction of a virus, such as the coronavirus 10, having an outer envelope or membrane 12 comprising generally lipid-type material and creating a boundary layer. Within the virus (inside 15 of the boundary layer 12) will exist an internal pressure Pᵢₙₜₑᵣₙₐₗ. Outside of the virus will exist an external pressure, Pₑₓₜₑᵣₙₐₗ. The intention of the methodologies disclosed herein are to physically disrupt the virus boundary layer 12 (membrane or greasy bubble) by subjecting the layer to a pressure differential, Δp, to an ultrasonic force or to a magnetic force sufficient to cause the boundary layer membrane 12 to rupture 12a. Under ambient, static conditions, Pᵢₙₜₑᵣₙₐₗ=Pₑₓₜₑᵣₙₐₗ=1 atm.

In view of the current understanding that the primary point of first entry of the coronavirus 10 into a human body (patient 30) is through an infection beginning in the lungs 32, one approach to the present disclosure is to safely subject a patient's lungs 32 to differing pressures to upset the static pressure equilibrium between the Pᵢₙₜₑᵣₙₐₗ and Pₑₓₜₑᵣₙₐₗ of the virus 10. A patient 30 infected with COVID-19 (coronavirus 10) residing at sea level has an internal lung air pressure of 1 atm (the atmospheric pressure at sea level). As such, it is logical to consider that the interior 15 of the greasy bubble (lipid bilayer 12) that surrounds the coronavirus 10 is also at an internal pressure of 1 atm at sea level. The object of the pressure differential methods of the present disclosure are to increase or decrease the air pressure inside the patient's lungs 32 to compress or expand the greasy bubble (membrane 12) that surrounds the coronavirus 10 and break this greasy bubble 12 with these pressure variations/pressure differentials, thereby destroying or disabling the coronavirus. Likewise, the object of the application of ultrasonic or magnetic energy (e.g., pulsed magnetic fields) to an infected area is likewise to cause a disruption in the functionality of the virus (or other pathogen) causing the infection or affliction, such as by damaging or destroying the membrane 12 of the virus 10 or the membranes important to functionality of other pathogens.

The coronavirus 10 is surrounded by a membrane or envelope layer 12 resembling a greasy bubble (thought to be a lipid bilayer). The coronavirus 10 lodges initially in the pulmonary alveoli. In one embodiment, the method of the present disclosure comprises the use of hyperbaric chambers to permit the patient 30 with coronavirus to be subjected to various pressure differentials within the patient's lungs 32 sufficient to disrupt the virus 10.

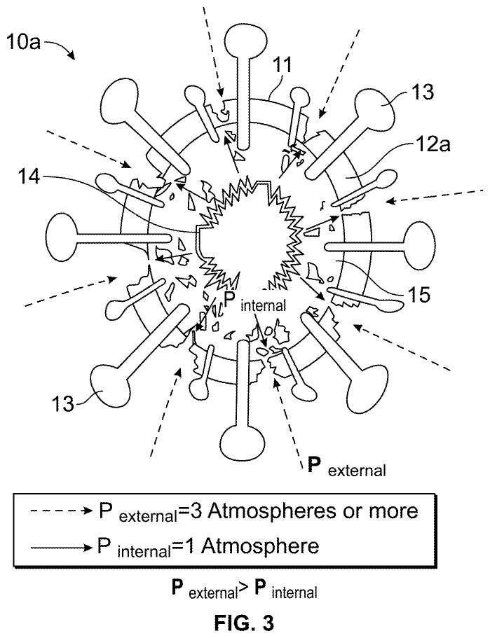

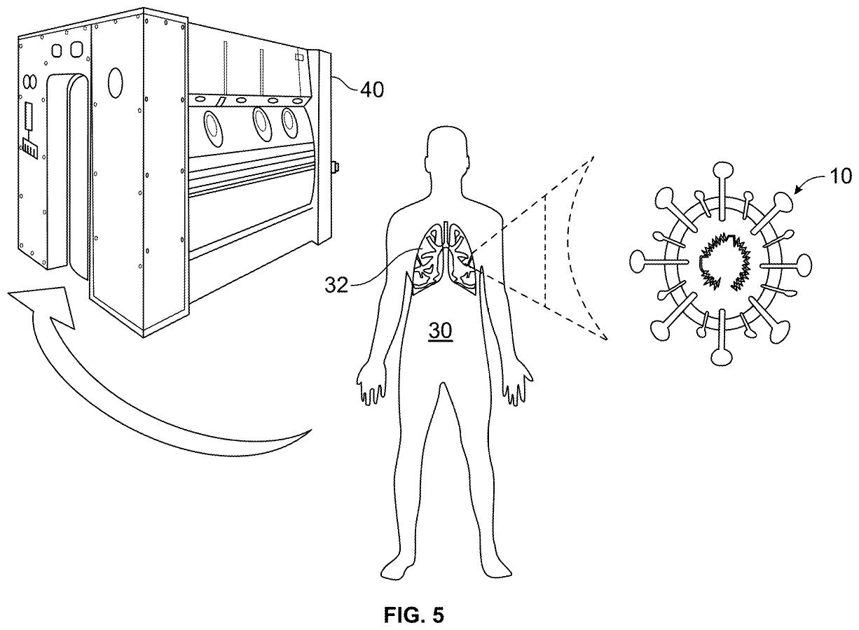

Pressure Differential Option 1: Referring now to FIGS. 3, 5 and 6, in one embodiment, a patient 30 with COVID-19 infection (or other viral infection) is placed into the interior 42 of a hyperbaric chamber 40 and subjected to pressure increases within the hyperbaric chamber in order to increase the air pressure inside the lungs 32 of the patient 30 and thereby achieve compression of the greasy bubbles 12 that surround the coronavirus 10 to the point that these greasy bubbles 12a rupture (by implosion) destroying the coronavirus 10a as illustrated in FIG. 3. In this embodiment, the external pressure exerted on the virus is greater than the internal pressure capacity of the virus, for example, 3 atm or greater (within the tolerances of the patient 30) thereby causing a destructive pressure differential across the virus's outer membrane layer 12a. In this embodiment, the treatment pressures, the rate of pressurization (e.g., incremental vs. continuous vs. cycling), and the duration of the pressurization would be monitored by medical professionals and would not exceed the pressures, rate of pressurization and duration of pressure that the patient's body could handle. In this embodiment, as may be necessary, the patient 30 might require ventilator assistance to ensure oxygenation of the lungs during the treatment time period when the lungs are exposed to this higher external pressure. As noted, the pressurization could be administered in continuous fashion, in bursts, or in cycles in an effort to best disrupt or disable the virus 10. In these embodiments, the desired external pressure can be applied in varying protocols, such as, steady pressure increases, pulsed pressure increases, or variations between pressure increases and pressure decreases, and the like. The methodologies of this option could likewise vary other conditions of the treatment environment, such as temperature, humidity, and the level of oxygen administered to the patient 30 (e.g., intervals of 100% oxygen interrupted with short periods of lower, or ambient oxygen levels).

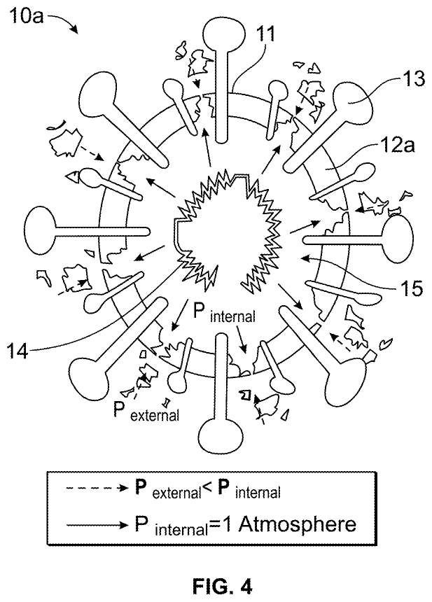

Pressure Differential Option 2: Referring now to FIGS. 4, 5 and 7, in another embodiment, a patient 30 infected with COVID-19 (or other viral infection) is placed into the interior 42 of a hyperbaric chamber 40 and subjected to a pressure decrease within the hyperbaric chamber in order to decrease the air pressure inside the lungs 32 of the patient 30 and thereby achieve expansion of the greasy bubbles 12 that surround the coronavirus 10 to the point that these greasy bubbles 12a rupture (by explosion) destroying the coronavirus 10a. In this second embodiment, the external pressure exerted on the virus 10 is less than the internal pressure capacity of the virus, thereby causing a destructive pressure differential across the virus's outer membrane layer 12a. In this embodiment, as may be necessary, the patient 30 might require ventilator assistance to ensure oxygenation of the lungs during the treatment time period when the lungs 32 are exposed to this lower external pressure. In these embodiments, the desired external pressure can be applied in varying protocols, such as, steady pressure increases, pulsed pressure increases, or variations between pressure increases and pressure decreases, and the like. The methodologies of this option could likewise vary other conditions of the treatment environment, such as temperature, humidity, and the level of oxygen (e.g., intervals of 100% oxygen interrupted with short periods of lower, or ambient oxygen levels).

Pressure Differential Option 3: Still with reference to FIGS. 3-7, a patient 30 who has already been subjected to treatment option 1 (i.e., an increase in external pressure within the hyperbaric chamber 40 to the limit that the human body can resist (e.g., 5 atmospheres, 10 atmospheres, etc.)), will now have the virus greasy bubble (external membrane) 12 that surrounds the coronavirus 10 reduced to its minimum possible size due to the external pressures. To the extent that the external pressurization did not rupture the virus membrane 12 (option 1), at this time, the patient 30 can be subjected to a rapid decrease (removal) of the pressure in the hyperbaric chamber (as can be safely handled by the limits of the human body) so that the internal pressure of the lipid bilayer 12 that surrounds the coronavirus 10 inflates (expands) rapidly thereby rupturing the external membrane 12a of the coronavirus 10a to the point that the virus explodes, destroying the coronavirus, or otherwise disrupting the virus' functionality. In these embodiments, the desired external pressure can be applied in varying protocols, such as, steady pressure increases, pulsed pressure increases, or variations between pressure increases and pressure decreases, and the like. The methodologies of this option could likewise vary other conditions of the treatment environment, such as temperature, humidity, and the level of oxygen (e.g., intervals of 100% oxygen interrupted with short periods of lower, or ambient oxygen levels).

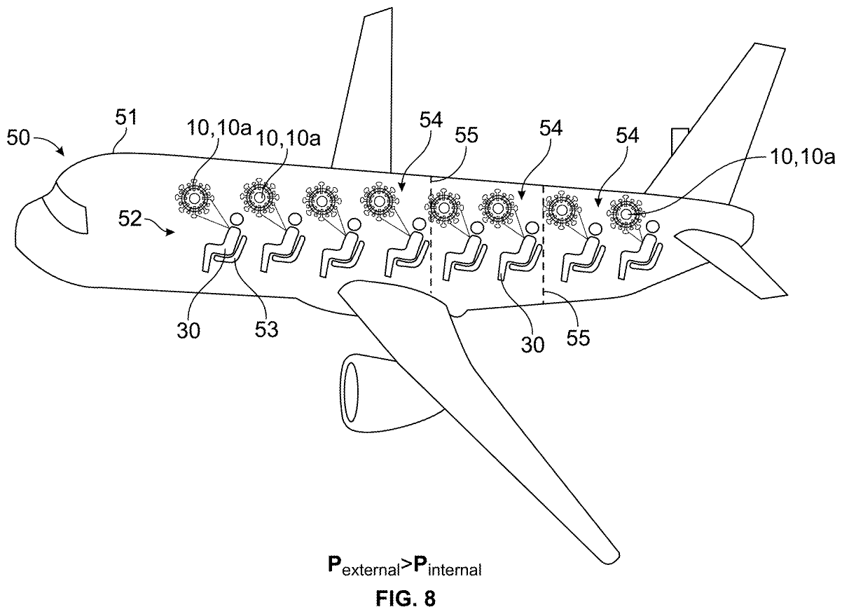

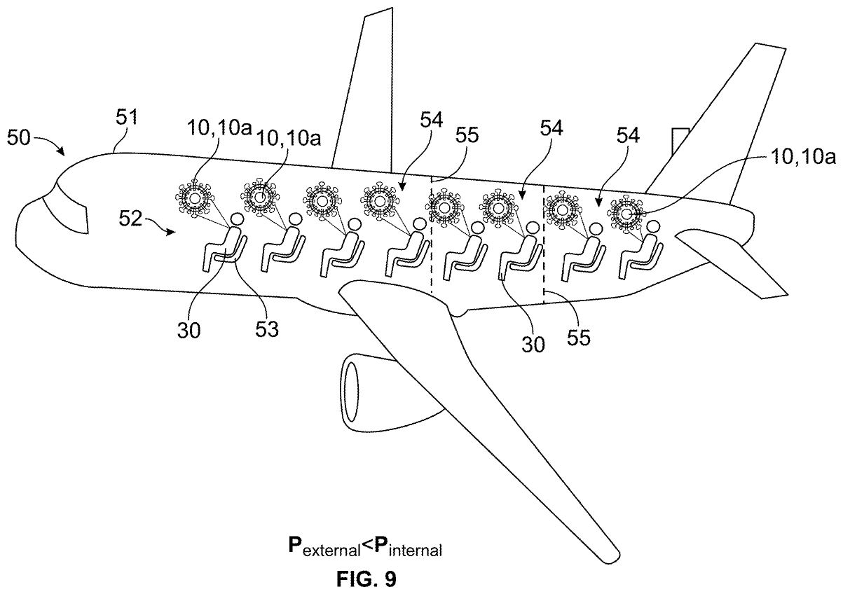

Pressure Differential Option 4: Referring now to FIGS. 8 & 9, in yet another embodiment, it is contemplated that the methods of options 1-3 can be achieved using a pressurizable air transport vehicle 50, such as a commercial jet aircraft, to create the desired external pressures by adjusting the pressure P_{cabin} of the pressurizable cabin 51 while the aircraft remains on the ground, or to transport the patient(s) up to high altitudes where the aircraft cabin pressure can be adjusted. At ground level, the air pressure is about 14.7 pounds per square inch thereby providing an appropriate amount of oxygen. Most commercial jets and private jets fly at an altitude between 30,000 feet and 40,000 feet. At these high altitudes, the air pressure is greatly reduced compared to levels on the ground. The lack of air pressure means that most humans will need oxygen masks above 18,000 feet due to the lack of oxygen. A sudden change in pressure, if an airplane descends or ascends too rapidly, can cause the loss of subconscious due to the excess nitrogen coming out of the bloodstream (barotrauma). To avoid this condition, airplane cabins 51 slowly and gradually depressurize and pressurize the cabin on landing and takeoff. At 35,000 feet, the air pressure is only about 4 pounds per square inch compared to 14.7 pounds per square inch. Most airlines are pressurized to maintain consistent air pressure between 11 psi and 12 psi. To avoid dangers associated with sudden changes in altitude, every plane has a pressurized system that replaces interior air with the air outside the cabin.

Although aircraft cabins 51 are pressurized, cabin air pressure P_{cabin} at cruising altitude is lower than air pressure at sea level. At typical cruising altitudes in the range 11,000-12,200 m (36,000-40,000 feet), air pressure in the cabin is equivalent to the outside air pressure at 1800-2400 m (6000-8000 feet) above sea level. As a consequence, less oxygen is taken up by the blood (hypoxia) and gases within the body expand. The effects of reduced cabin air pressure are usually well tolerated by healthy passengers.

The medical hyperbaric oxygen therapies for COVID-19 patients are preferably carried out at about 2 atmospheres of pressure, that is, 29.38 PSI. At ground level, the ambient air pressure is a little over 14 PSI, and at a typical aircraft cruising altitude (e.g., 30,000 to 40,000 feet) the air pressure may be just 4-5 PSI. Aircraft cabins are pressurized to simulate pressure felt at 8000 feet. The pressure differential in an airplane in flight (internal vs external pressure) varies up to 9 PSI. Then from the ground pressure 1 ATM=14.69+9.00=23.69 PSI. Therefore, an aircraft can easily make pressure variations from 14.69 PSI up to 23.69 PSI. To achieve the desired 2 atmospheres of pressure for the disclosed hyperbaric oxygen treatment in a pressurized aircraft cabin, we are missing 29.38 PSI−23.69 PSI=5.69 PSI. The air pressure of the aircraft is supplied by the compressors that the aircraft has in the aircraft's engines. Typically for an aircraft in flight, pressurization of the cabins takes place via the aircraft's jet engines compressing incoming air, heating it, and then diverting some of it to the cabin (after first having been cooled and humidified using climate control systems) until the desired cabin pressure is met. The pressure is typically maintained by an outflow valve. These aircraft cabin pressure regulation systems (or cabin pressure control and monitoring systems as are well known in the aerospace industry) as well as the aircraft's fuselage, are designed with very high safety margins, therefore, an aircraft's cabin can be pressurized to 2 Atmospheres of Pressure, even more so considering the embodiments described herein that utilize the aircraft positioned on the ground (not in flight). It is during an aircraft's flight when the engines and the fuselage are subjected to great extraordinary efforts due to the altitude, aircraft speed, etc.

Alternatively, the aircraft 50 could be outfitted with one or more separate climate-controlled treatment rooms or treatment zones 54 located within the airplane fuselage 51 that are capable of independent pressure adjustment. The present disclosure therefore also contemplates the possibility of using airplane cabins, or chambers within airplane cabins for use in subjecting the patient(s) to the aforementioned pressure differentials. In one example of this embodiment, a group of coronavirus patients are brought aboard a plane. Before taking off, one can assume that the air inside the lungs of these patients is at a 1 atm, and the interior of the greasy bubbles (external membranes) that surround the coronavirus in the patient's lungs are also at a 1 atm. The cabin can then be pressurized for the comfort of the passengers, and the airplane can take off and ascend to a height of 30,000 ft above sea level. At that time, in a controlled manner, the pressure in the cabin (or in a separate treatment unit within the cabin) can be reduced. Preferentially, the drop in pressure will be controlled enough to reduce the risk of rupturing the patient's eardrum, or creating other health issues, while at the same time being of a sufficient pressure differential to cause the outer membrane of the coronavirus to rupture due to the rapid expansion of the internal pressure of the virus' external membrane. Thus, in one embodiment, one or more virus infected patients are transported in an airliner to sufficient altitudes to permit the manipulation of cabin pressure (or pressure within a treatment unit in such aircraft) sufficient to disrupt the outer membrane of the virus. In one embodiment, a traditional airliner is employed, and the patients 30 are located in existing seats 53, and are accompanied by medical personnel 38 and any required ancillary medical equipment, such as oxygen 71, ventilators, etc. In another embodiment, an airliner is specially outfitted with one or more single- or multi-person treatment chambers that can permit separate manipulation of the ambient pressures therein, while the airliner is flying at desired altitudes. In these embodiments, the desired external pressure can be applied in varying protocols, such as, steady pressure increases, pulsed pressure increases, or variations between pressure increases and pressure decreases, and the like. The methodologies of this option could likewise vary other conditions of the treatment environment, such as temperature, humidity, and the level of oxygen (e.g., intervals of 100% oxygen interrupted with short periods of lower, or ambient oxygen levels).

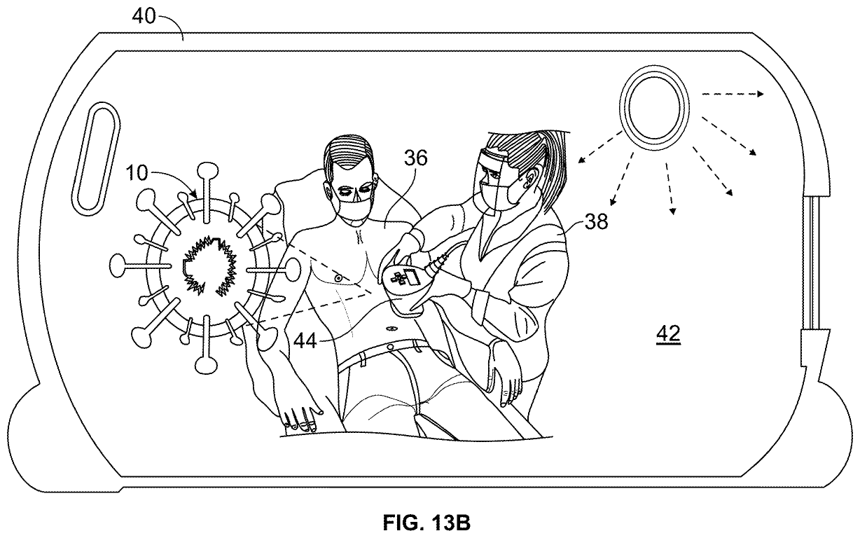

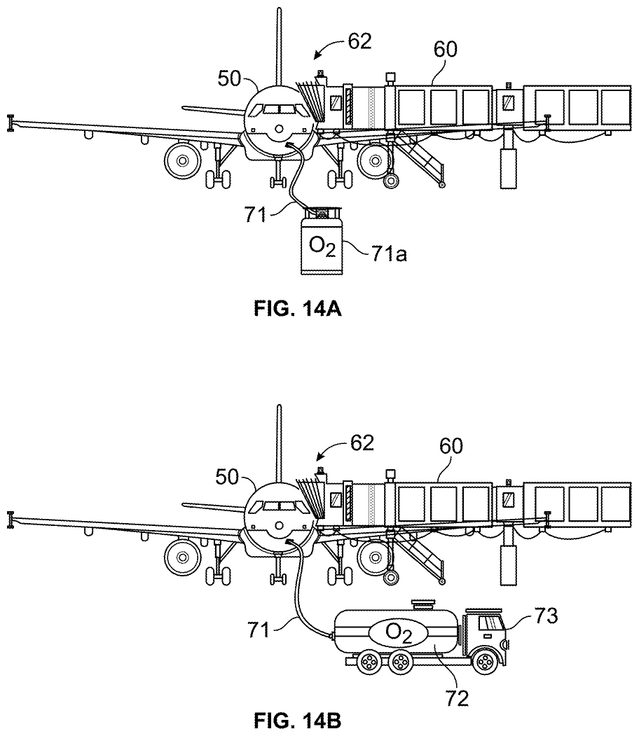

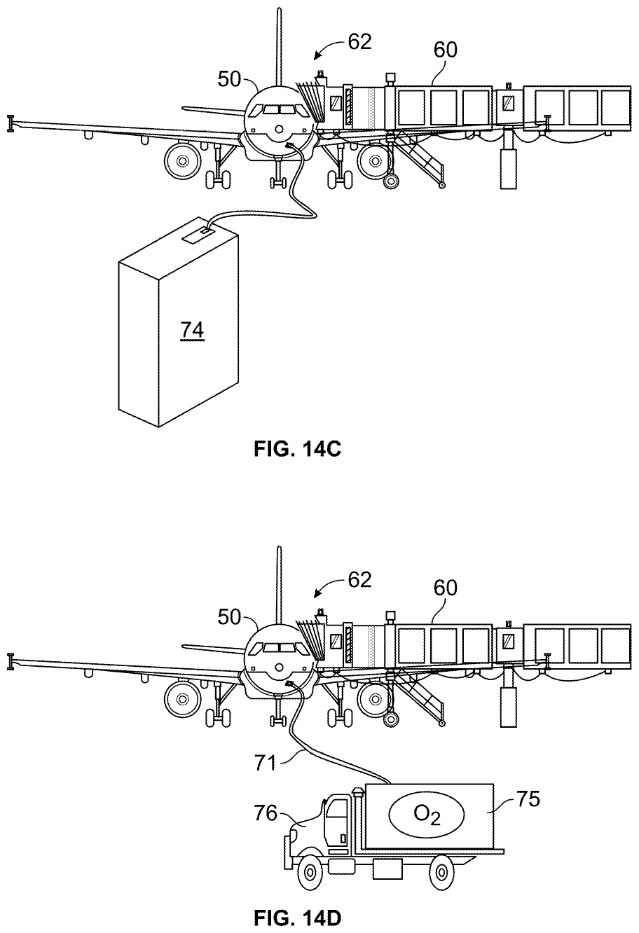

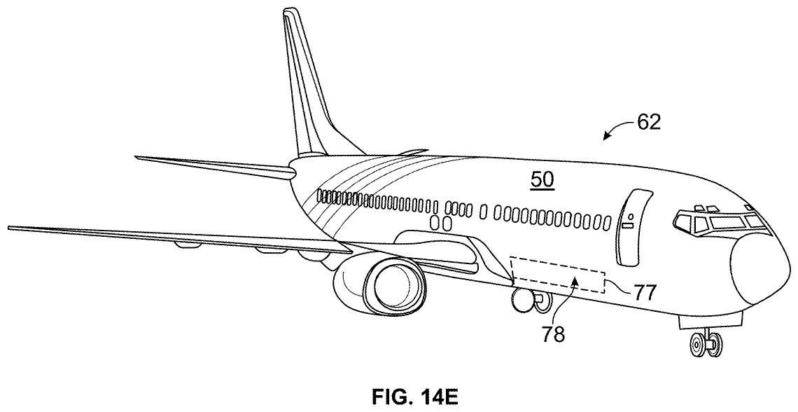

Referring now to FIGS. 14A-14E, 15A, 15B, 16A, 16B, 17A, 17B, and 18A-18C, there are shown various embodiments of the present disclosure utilizing a stationary aircraft 50 (or multiple aircraft) located on the ground for use as an HBOT treatment center.

For example, FIGS. 14A and 14B schematically depict an aircraft 50 stationed on the ground wherein the pressurizable cabin 51 will be used as a hyperbaric chamber or HBOT treatment center 62 according the disclosures herein. In this embodiment, the aircraft 50 utilizes its own engine power to, e.g., pressurize the cabin 52 and provide power, but is provided with an external source of medical grade oxygen 71 for use within the cabin, preferably to be administered to each patient 30 via individually regulatable masks 70 worn by the patients 30 on the aircraft 50. Although commercial aircraft already have an emergency oxygen supply that can be deployed via masks 70, the standard onboard oxygen supply of a commercial aircraft is of a limited use quantity designed to provide oxygen on an emergency basis during those situations where the aircraft loses cabin pressure and must therefore safely descend to a lower altitude. During descent, the passengers use their deployed oxygen masks. Once the aircraft has reached a lower safe flight altitude, it can again rely on the ambient oxygen level of the external air to provide safe levels of oxygen in the cabin. Thus, the oxygen supply for each passenger on a traditional commercial airliner will likely not provide the desired length of HBOT oxygen treatment while the aircraft 50 is being used as an HBOT treatment unit 62. Therefore, in this embodiment, an external source of medical grade oxygen 71 may be provided by a stationary tank of pressurized medical grade oxygen (gas or liquid state) 71a located proximate the aircraft 50 or be provided by a mobile vehicle 73 providing the tank source of oxygen 72.

Similarly, FIGS. 14C and 14D reflect the same as in FIGS. 14A and 14B, except that the source of stationary oxygen 71 shown in FIG. 14C is provided by a stationary oxygen concentrator or generator 74 located proximate the aircraft 50 or as provided by a mobile vehicle 76 providing the oxygen generator 75. It will be understood by those with knowledge in the art that many medical grade oxygen systems and oxygen generation systems exist commercially for providing or generating the high volumes of medical grade oxygen required, e.g., those used by hospitals, and such systems could be employed to supply the required oxygen requirements for the HBOT treatment center 62, either in a mobile unit or in a permanently installed unit. The oxygen supply could be tied in with the aircraft's existing network of oxygen masks, or the aircraft could be modified to house its own oxygen distribution network to route the oxygen to the desired end locations much like with gas distribution systems used in hospitals.

FIG. 14E illustrates where a commercial aircraft 50 could be retrofitted with its own onboard oxygen concentrator 77 located, e.g., within the cargo hold area 78 of the aircraft 50. In these embodiments, the supplemental source of generated medical grade oxygen is connectable to the aircraft's existing oxygen system to permit the distribution of medical grade oxygen therethrough. Alternatively, an independent oxygen delivery system could be installed or retrofitted, independent of those found in existing aircraft, to distribute oxygen to desired locations of the aircraft's cabin 52, e.g., to each seat 53 or patient treatment location/zone 54 or treatment station.

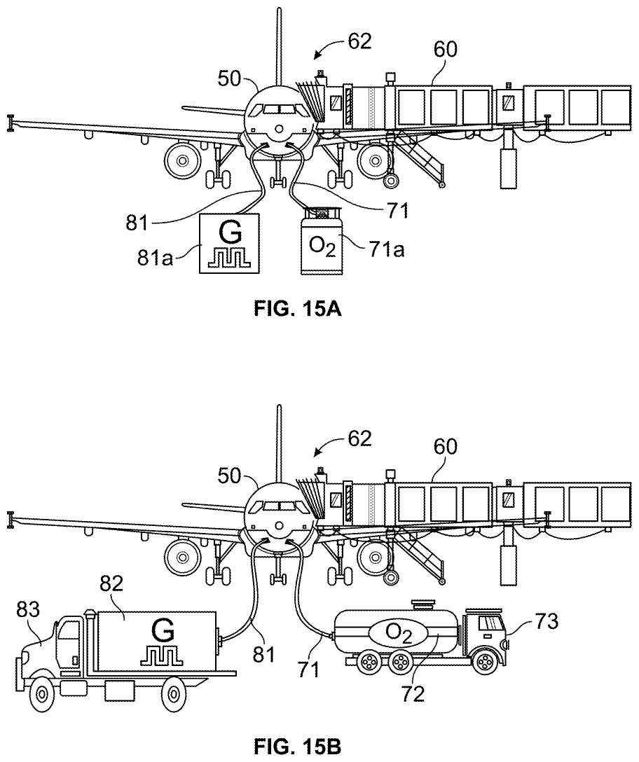

FIGS. 15A and 15B are similar to FIGS. 14A and 14B, but add the use of external power supply 81 provided via stationary power generation systems 81a located proximate the exterior of the aircraft 50 or the use of a mobile power generation system 82 mounted on a vehicle 83. In these embodiments, the external source of power 81 is connectable to the aircraft's existing power system to permit the distribution of power therethrough.

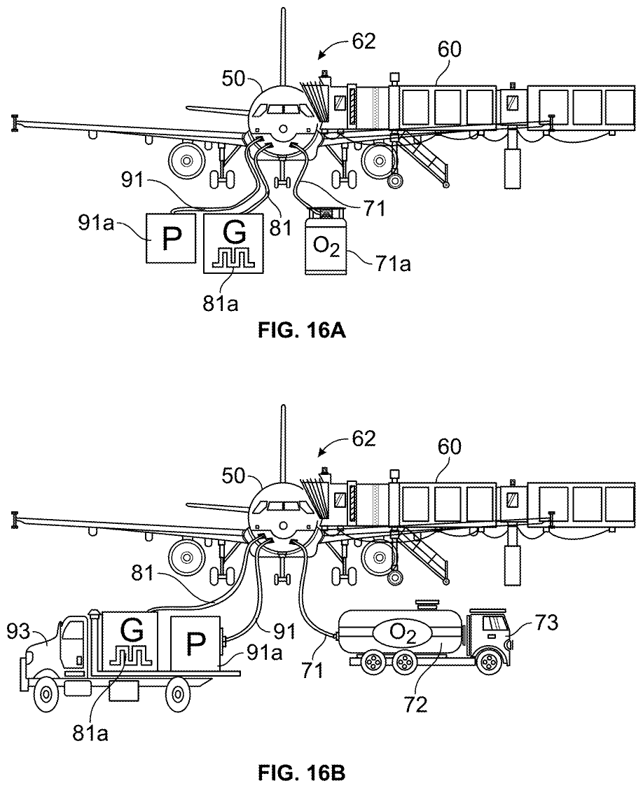

FIGS. 16A and 16B are similar to FIGS. 15A and 15B, but add the use of an external source of cabin pressurization 91 delivered either via a stationary pressure generator 91a located proximate the exterior of the aircraft 50 or the use of a mobile cabin pressurization system 92 mounted on a vehicle 93. In these embodiments, the external source of cabin pressurization 91 is connectable to the aircraft's existing cabin pressurization system to permit the distribution and regulation of pressure therethrough.

Similarly, an external climate control system (e.g., heater, air conditioner, humidifier, etc.) (not shown) may be employed to provide the desired climate within the cabin or within portions of the cabin.

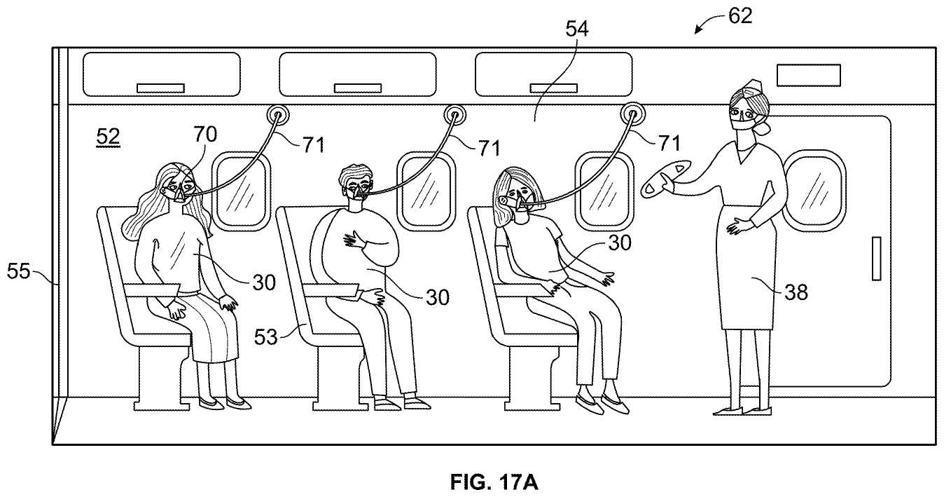

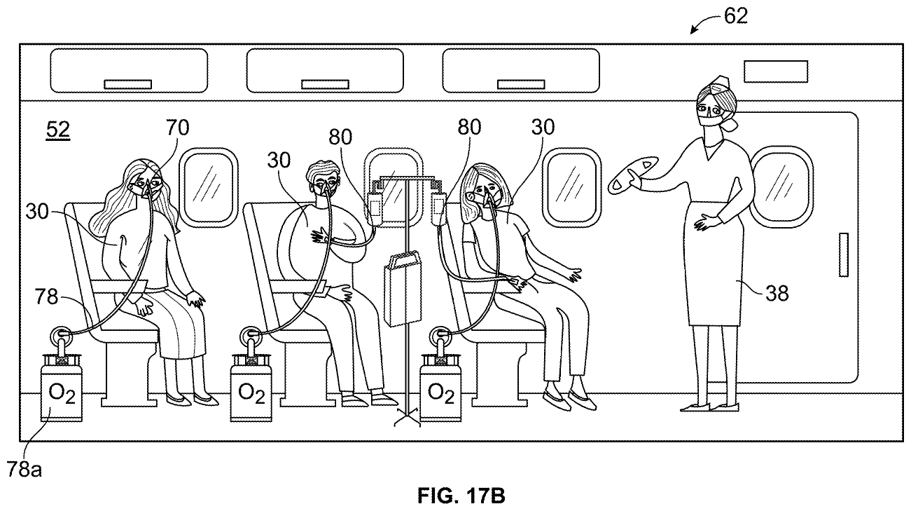

Referring now to FIGS. 17A and 17B there is depicted a portion of the interior of an aircraft cabin 52 being used in connection with the various treatment regimens described herein. For example, each patient 30 is seated in seats 53 and receiving medical grade oxygen 71 or other desired treatment gases via masks 70 from the external oxygen delivery system (FIG. 17A) or is receiving medical grade oxygen 78 from individual portable oxygen concentrators 78 (FIG. 17B). As may be desired, patients can also receive IV fluids 80 or the like as may be desired or required. Various medical personnel 38 are also present to attend to the patients 30. In a preferred embodiment, each individual patient's mask 70 is capable of being controlled separately as to the gas volume and gas composition passing therethrough via a suitable regulator (not shown) such as might be found in a hospital setting. For example, patients may be administered 100% oxygen interrupted at desired intervals by a lower oxygen content. Other patients may be administered supplemental gasses as described herein or as otherwise medically desired. Various patient monitoring equipment can also be employed as would be found in a hospital, emergency room, or intensive care unit. FIG. 17B shows an alternative source of medical grade oxygen 78 being supplied by portable oxygen concentrators 78a located proximate the patients 30. In one embodiment, each patient treatment station employs a portable oxygen concentrator as are known in the art. These portable oxygen concentrators may be used for individual patients, or used to provide oxygen to multiple patients.

Existing aircraft are capable of creating an internal cabin pressure of, e.g., 2 ATM, are already outfitted with large seating capacities (e.g., 300 seats 52), have climate control, and have existing oxygen delivery systems along with oxygen masks available for use at each seat. This makes existing aircraft ideal for rapid adaption and deployment for use as large-scale HBOT treatment units capable of treating many patients at a time. Each aircraft 50, whether used for on-ground treatments or in-flight treatments could be outfitted as desired to create one or more isolatable treatment zones 53, for example, by installing plastic curtains 54 or the like. More serious patients could be treated in a separate zone. Patients with less or non-serious conditions could be treated in another section. In one embodiment, the patients enter the aircraft 50 on the ground via one or more passenger boarding bridges (jet bridge or jetway) 60, boarding ramps or stairs or other suitable passenger boarding systems along with the requisite medical professionals. The patients 30 are then seated in seats 52 and provided with oxygen masks 70. Medical grade oxygen is provided to the patients' masks (and as may be desired, each seat can be outfitted with the ability to control the specific level of oxygen being provided to each patient's mask). The cabin doors are closed and the aircraft cabin 51 is pressurized to the desired cabin pressure for the duration (e.g., 90 minutes) of the treatment. The cabin 51 is then depressurized, and the patients 30 exit the aircraft 50. The aircraft 51 can then be quickly readied for next use by using appropriate disinfection protocol, including changing out the masks. In this embodiment, the aircraft remains stationary on the ground, and uses an external tank source of oxygen 71 that can be replenished as needed (e.g., a stationary large tank of oxygen 71a, or a large portable tank of oxygen 72, or large-scale oxygen concentrator 75. With these large external oxygen tanks or oxygen concentrator, enough oxygen can be provided to treat hundreds of patients in a day. In another embodiment, the oxygen 78 is provided via portable oxygen concentration units 78a for each seat (or group of seats) capable of providing medical grade oxygen.

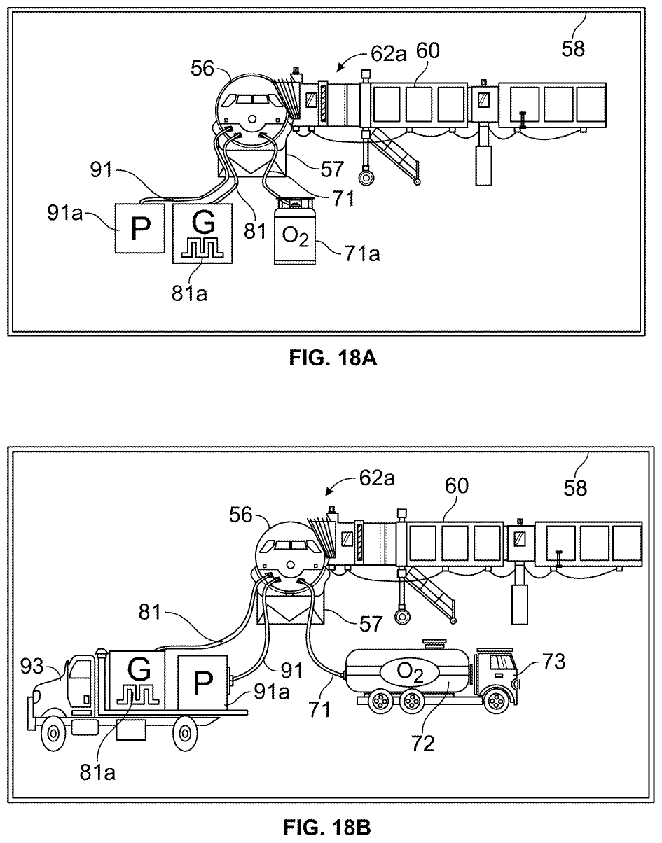

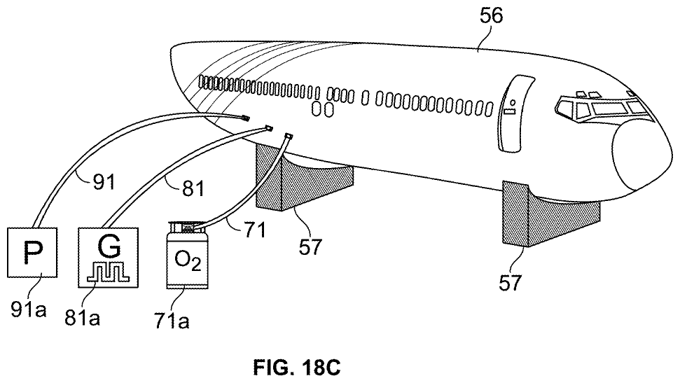

Likewise, retired aircraft can be used as on-ground HBOT treatment units 62, even if they are no longer flight worthy. In one embodiment, the retired aircraft 50 can be flown to a final destination for use, and if desired for purposes of reducing space requirements, as depicted in FIGS. 18A-18C, the wings and engines of the now retired aircraft can be removed and the tubular fuselage 56 can be outfitted as needed with external cabin pressurization equipment, power supply, climate control equipment, and oxygen supply to create large, multi-patient HBOT treatment units 62a. Multiple aircraft cabin fuselages 56 could be co-located together to create a treatment facility with large treatment capabilities. For convenience, these aircraft fuselage 56 may ideally be located within a climate-controlled space 58.

Although it is envisioned that an existing aircraft can create the desired internal cabin treatment pressure while positioned on the ground, it is also envisioned that if necessary or desirable, when an aircraft's cabin is used as a stationary HBOT treatment center 62 on the ground, it could be outfitted (retrofitted) with supplemental cabin pressurization systems if desired that operate independently of the aircraft's engines.

Referring now to FIGS. 18A, 18B and 18C, there is depicted one or more airplane fuselages 56 that may be employed as described herein to provide a pressurizable, climate controllable cabin for carrying out the treatment methodologies described herein. In these stationary HBOT treatment center 62a embodiments, to conserve space, the aircraft's wings and engines have been removed, and its wheels have been replaced with permanent fuselage mounting structures 57. These fuselages 56 may be housed within a climate-controlled building 58 to reduce exposure to outside elements. Appropriate entrances, such as a jetway like entrance 62 or the like would be used to permit patients to enter the HBOT treatment center 62a.

As an alternative to the use of a hyperbaric chamber or mobile pressurizable treatment unit, and in connection with the potential use of a ventilator, the pressurization of the patient's lungs could be achieved via the use of a continuous positive air pressure (CPAP), positive air pressure (PAP), BIPAP (bilevel positive airway pressure), or other means for administering pressure to the airways, such as via a non-invasive helmet system. Most CPAP machines have a pressure setting range from about 6 cm/H2O to 15 cm/H2O (measured in centimeters of water pressure) with the average typically 10 cm/H2O. Specialized CPAP machines can deliver CPAP pressure up to 25 to 30 cm H2O. It is envisioned, however, that for purposes of the present disclosure, a CPAP, BIPAP, PAP, or helmet device could be modified accordingly to deliver the desired treatment pressures. In these treatment regimens, the pressure could be applied in pulses to create the pressure differentials across the virus membrane to cause the virus membrane to rupture or to otherwise interfere with the virus's functionality.

As with all of these treatment methodologies, the employment of pressure differentials on the patient would be conducted with as much control as possible so as to avoid causing any collateral damage to the patient, such as, rupturing of eardrums. For example, in typical Hyperbaric Oxygen Treatments, the air pressure in the chamber is often 1.5 to 3 times greater than ambient air pressure. Most typical indications for HBOT involve the use of hyperbaric pressures above 2.0 atm. Higher atmospheric pressures are generally required to treat conditions such as carbon monoxide poisoning and to improve wound healing. The pressure increases in the present treatment methods are envisioned to range from greater than 1 atm to about 7 atm, again depending on the general physical condition of the patient. When applying the increased pressure treatments, it is preferred to gradually increase the pressure up to the maximum desired value. When placing a patient in a vacuum or partial vacuum atmosphere, it is likewise envisioned that 0.5 atm, 0.4 atm, etc. could be utilized according to the tolerances of the patient's body.

In addition to the use of pressure differentials to destroy the virus, it is also contemplated that these pressure differentials can also cause interference with the virus' ability to effectively interact with the ACE2 receptor functionality. It is also contemplated that these pressure differentials could potentially enhance the effectiveness of statins or ACE2 inhibitors or otherwise reduce risks associated with blood clotting that has been experienced in patients infected with COVID-19. Furthermore, it is also contemplated that these pressure variations can disrupt the virus' ability to adversely affect endothelial cell functions. It is also contemplated that the use of these pressure differentials could enhance other treatment regimens used to combat the virus in an infected human.

Although many of the teachings herein focus on the treatment of patients experiencing an active infection, the methodologies taught herein have equal applicability to post-infection therapy to treat or minimize the occurrence of post-infection syndrome. It is further envisioned that the treatment methods described herein could employ variations in the oxygen levels administered during the HBOT treatment, for example, by providing the patient with intervals of 100% oxygen followed by intervals of ambient air.

Furthermore, it is likewise envisioned that the hyperbaric chamber described herein could comprise a pressurized body suit (not shown) worn by the patient.

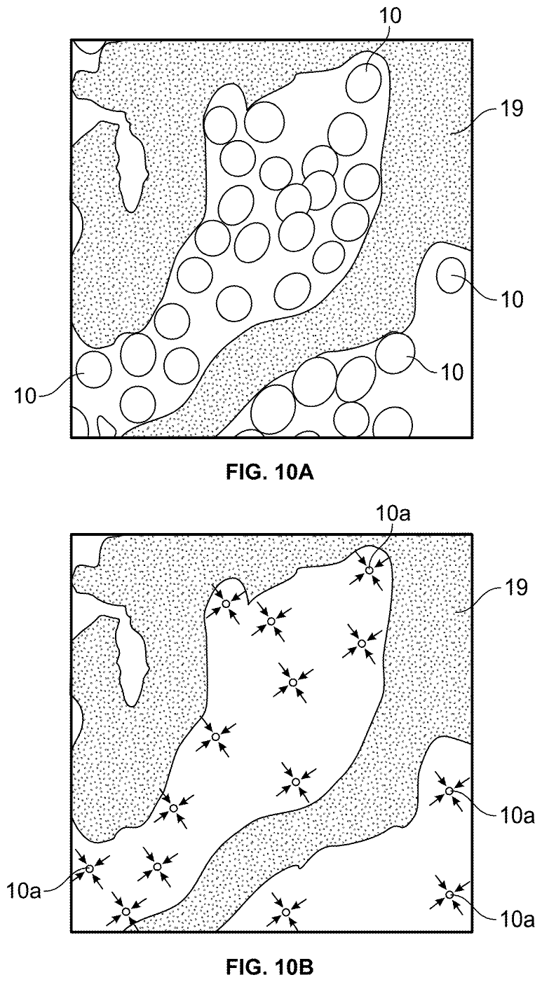

Referring now to FIG. 10A, there is schematically depicted a set of coronaviruses 10 penetrating adipose tissue 19 from a sample collected from a patient. Referring now to FIG. 10B, it is envisioned that when the virus infection treatment methods of the present disclosure are applied to a patient, the virus 10a should become destroyed or rendered inactive as depicted.

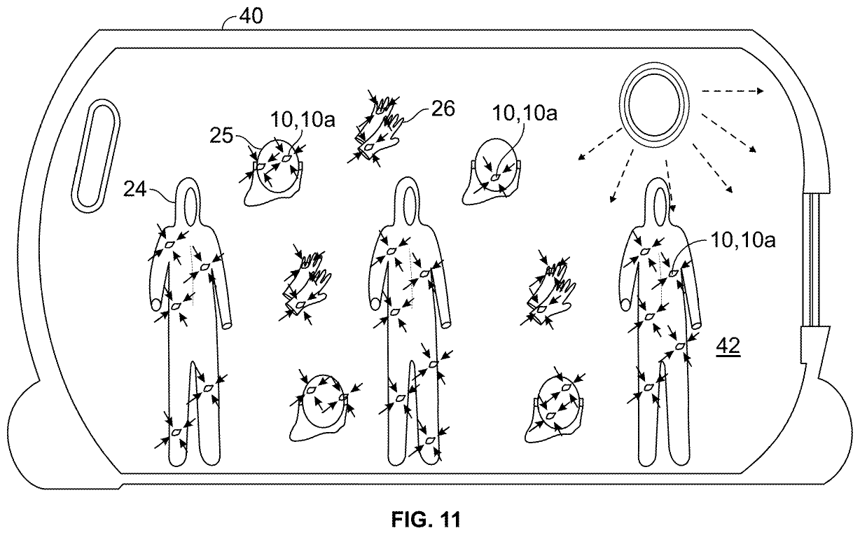

Referring now to FIG. 11, there is shown a schematic representation of another treatment method of the present disclosure utilizing a hyperbaric chamber 40 to apply a desired external pressure to personal protective equipment (PPE), such as, masks, gloves, goggles, jumpsuits, or other PPE in need of sterilization against potential contamination from, e.g., a virus, such as coronavirus or other biological contaminant. In one embodiment, the virus is thereby exposed to an external pressure greater than the internal pressure of the virus, thereby causing a pressure differential sufficient to cause the destruction of the virus (or its protective outer layer), to disable the virus, or to otherwise interfere with the virus functionality. In another embodiment, the virus is thereby exposed to an external pressure lesser than the internal pressure of the virus, thereby causing a pressure differential sufficient to cause the destruction of the virus (or its protective outer layer), to disable the virus, or to otherwise interfere with the virus functionality. It is envisioned that most medical equipment can tolerate great positive or negative pressures, e.g., from an absolute vacuum (0.0 atm) to 50 to 100 atm or more. Likewise, most medical equipment can be subjected to very rapid, and dramatic pressure changes that would otherwise not be tolerated by a virus' outer membrane 12.

Referring now to FIG. 12, there is depicted a schematic representation of an ultrasonic cavitation procedure using an ultrasound device 44, such as that used for, e.g., aesthetic, non-invasive fatty tissue removal being performed in a hyperbaric chamber 40 to apply a desired external pressure to a human patient 34 undergoing such procedure to enhance the effectiveness and efficiency of the ultrasonic cavitation or ultrasound procedure. These “spa-like” treatments could be carried out in a large, multi-person hyperbaric chamber, such as for example, one created from the cabin of a retired commercial aircraft as described herein. In this embodiment, the aircraft cabin could be partitioned into individual treatment rooms to permit patient privacy. Large numbers of patients could be treated during a single pressurization interval. The patients could also be administered medical grade oxygen at desired concentrations to enhance the treatment.

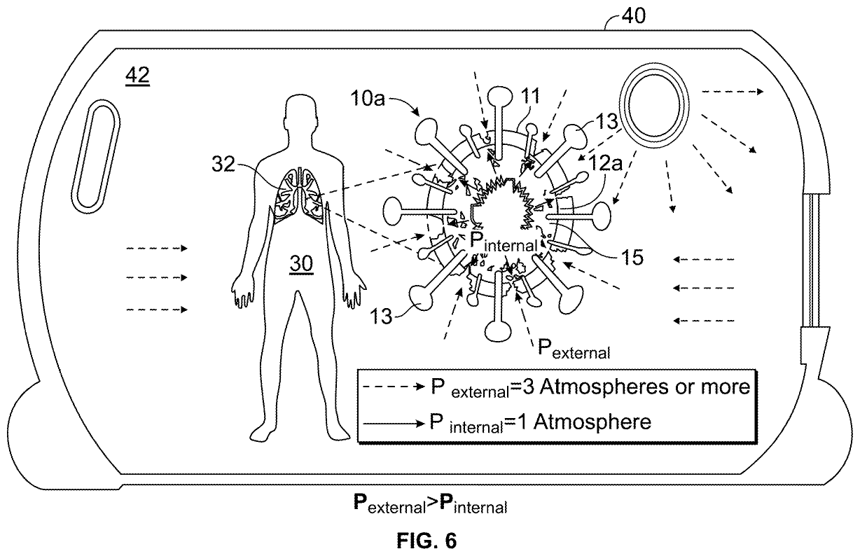

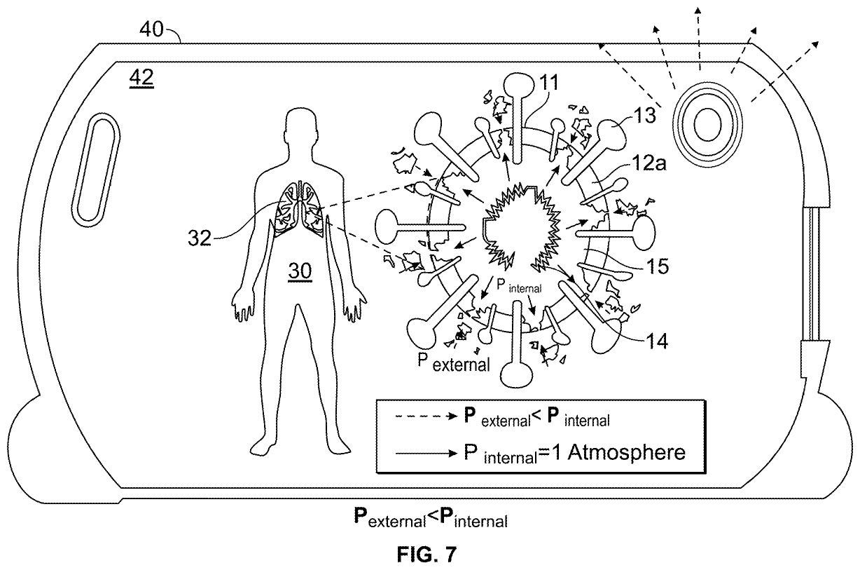

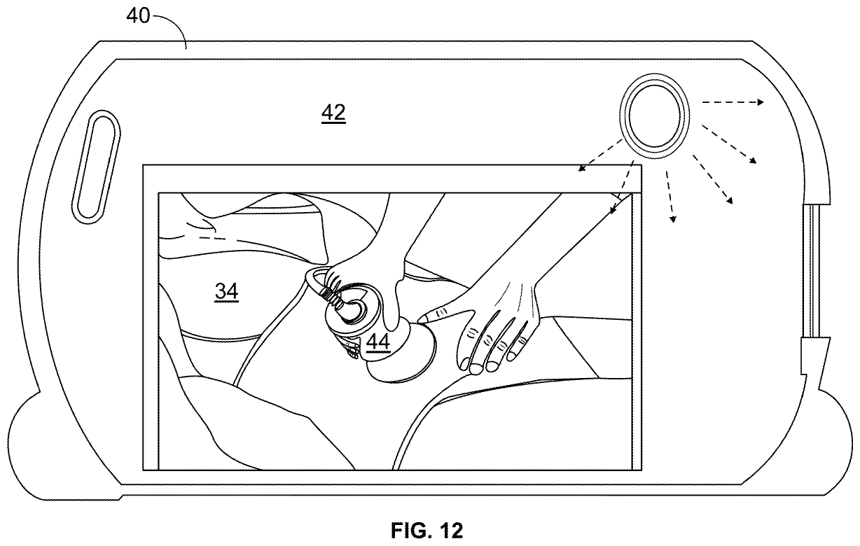

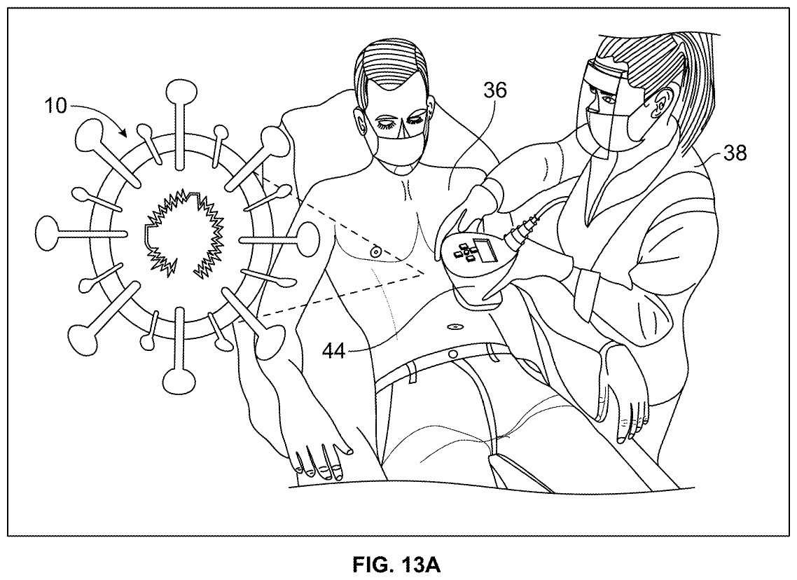

Referring now to FIG. 13A there is shown a schematic representation of an ultrasonic/ultrasound cavitation procedure being applied to a human patient's 36 external torso in the region of the lungs to assist in destruction or disabling of a virus such as the coronavirus infecting the patient's lungs according to another embodiment of the present disclosure. The ultrasound procedure could be conducted with standard ultrasonic/ultrasound cavitation equipment 44, portable ultrasound equipment, and if desired, focused ultrasound coupled with MRI (not shown) to assist the medical personnel in identification of the location of the virus-infected tissue and the monitoring of the virus destruction. Referring now to FIG. 13B in connection with FIGS. 6 and 7, there is shown a schematic representation of an ultrasonic cavitation or ultrasound procedure such as in FIG. 13A being applied to a human patient's external torso in the region of the lungs to assist in destruction or disabling of a virus such as the coronavirus infecting the patient's lungs according to another embodiment of the present disclosure. In this embodiment, this cavitation procedure is performed within a hyperbaric chamber 40 under increased or decreased external pressure as described in connection with FIGS. 6 and 7, including providing the patient with oxygen at desired concentrations and intervals.

Thus, the mechanosensitive nature of the coronavirus can provide a unique opportunity to design ultrasonic therapies that target infected tissue and disrupt or disable the virus. In this respect, recent studies have shown that the Inventors' proposed ultrasonic (ultrasound) cavitation method for treatment of humans infected with a virus such as one in the coronavirus family, has theoretical promise as a means for deactivating or disrupting the coronavirus' functionality. For example, researchers at MIT (Wierzbicki et al.) recently published research concluding that simulations show that ultrasound waves at medical imaging frequencies can cause the virus' shell and spikes to collapse and rupture. See, Tomasz Wierzbicki, Wei Li, Yuming Liu, Juner Zhu, Effect of receptors on the resonant and transient harmonic vibrations of Coronavirus, J. Mechanics and Physics of Solids, Vol. 150, 2021, 104369, ISSN 0022-5096, https://doi.org/10.1016/j.jmps.2021.104369. (https://www.sciencedirect.com/science/article/pii/S0022509621000600)(incorporated herein by reference). Focused ultrasound has also recently been proposed for use in treating patients with Parkinson's disease. See Martinez-Fernández et al. Randomized trial of focused ultrasound subthalamotomy for Parkinson's disease, N Engl J Med December 2020; 383:2501-2513 DOI: 10.1056/NEJMoa2016311. The use of ultrasound or ultrasonic cavitation procedures for treatment of the active virus could also be enhanced with the combined use of focused ultrasound and MRI to permit focused therapies in the regions of the body requiring the treatment. See also Arvanitis et al., Combined ultrasound and MR imaging to guide focused ultrasound therapies in the brain. Phys. Med. Biol. 58 (2013) 1-13; and Sirsi S R, Fung C, Garg S, Tianning M Y, Mountford P A, Borden M A. Lung surfactant microbubbles increase lipophilic drug payload for ultrasound-targeted delivery. Theranostics. 2013; 3(6):409-419. Published 2013 May 20. doi:10.7150/thno.5616 (noting that at higher ultrasound intensities, excess lipid was observed to be acoustically cleaved in connection with targeted drug release therapies). It is preferable to use the ultrasonic therapies of the present disclosure in a guided (focused) fashion so that the unhealthy tissues could be targeted while avoiding to the greatest extent possible, the healthy tissues. Further, the ultrasonic methods of the present disclosure could also employ the use of nanoparticles or nano-bubbles to target the infection-damaged tissue. The use of low-intensity pulses of ultrasound can target the infected tissue.

Additionally, although the above pressure differential methods have focused on creation of pressure differentials across the virus membrane, it is envisioned that other treatment regimens could be utilized in parallel to enhance the potential for rupture or disabling of the virus. For example, given that one of the primary points of introduction of the virus is through the respiratory system, perhaps any number of influencers could be used with pressure. For example, most fluid-like “bubble” structures have a certain surface tension that causes the layer to behave somewhat like an elastic sheet, there being an optimal surface tension for bubble stability. It is therefore envisioned that factors external to the virus can be introduced, e.g., into the lungs, that can disrupt the surface tension of the virus wall to enhance the effects of the pressure differential and thereby enhance the likelihood of the virus membrane rupturing or becoming disabled. For example, temperature and humidity could be varied and optimized to place the virus membrane at highest risk of rupture. Also, use in connection with known inhalers, such as those used in the treatment of asthma and COPD, e.g., corticosteroid inhalers (such as fluticasone), and bronchodilator inhalers (such as albuterol) and those inhalers infused with nasal decongestants (including natural menthol and camphor-based products) might assist in enhancing the vulnerability of the virus membrane to rupture. Other natural inhalable substances, e.g., herbal inhalers, aromatherapeutics, etc. could be used in connection with the pressure treatments. Additionally, treatments used for clearing mucus plugs in the lungs might be useful in altering the virus surface tension to promote rupture or disabling of the virus membrane by the desired pressure differential.

For example, inhalable medical-food grade alcohol (EtOH) or the like, e.g., administered as a vapor, (at desired temperatures), as nebulized aerosol, mist, alone or in combination with air or oxygen, etc. into a patient's lungs in a controlled treatment regimen could also have a disruptive impact on the functionality of exposed virus located within the patient's lungs and serve as a valuable treatment regimen against COVID-19 or other virus located in the lungs. Use of this form of treatment, under controlled conditions, is thought to address any known concerns about this type of alcohol consumption becoming addictive. Research suggests that a controlled treatment with inhalable alcohol (as opposed to recreational use of the same) poses a negligible health risk associated with alcohol inhalation. See, e.g., MacLean R R, Valentine G W, Jatlow P I, Sofuoglu M. Inhalation of Alcohol Vapor: Measurement and Implications. Alcohol Clin Exp Res. 2017; 41(2):238-250. doi:10.1111/acer.13291. As such, vaping of EtOH vapor (or other administration of EtOH vapor into the lungs) has application as a means for contacting and disrupting a virus present within a patient's lungs. In this embodiment, the EtOH vapor could be delivered to the patient through suitable delivery methods, e.g., ventilator, mask, inhaler, etc. This methodology could be used alone or in combination with other methods described herein, e.g., during HBOT treatment, etc. See also, Feinberg, Alec. (Jul. 14, 2020). The Use of Alcohol Inhalation in Ventilators or Other Suitable Apparatus for the Treatment of Covid-19. 10.13140/RG.2.2.30657.38248/2.

It is further envisioned that the treatment methods described herein could employ variations in the oxygen levels administered during the HBOT treatment, for example, by providing the patient with intervals of 100% oxygen followed by intervals of lower oxygen levels, such as those in ambient air. These high oxygenation intervals followed by intervals of rapidly decreased oxygen are thought to enhance the overall effectiveness of HBOT therapies.

There is also disclosed a method of treating a living subject infected with a virus or other pathogen by the administration of magnetic forces, such as those provided by Magnetic Resonance Imaging (MRI) magnetic fields, pulsed magnetic fields, rotating magnetic fields, alternating magnetic fields, oscillating magnetic fields, dynamic magnetic fields, magnetic nanoparticles, and magnetic hyperthermia. Use of MRI and magnetic fields avoids the safety concerns encountered with radiotherapy. During the COVID-19 pandemic, medical professionals relied on MRI to provide scans of, e.g., a patient's lungs as a tool for the diagnosis of the COVID-19 infection. See, e.g., Vasilev Y A, Sergunova K A, Bazhin A V, Masri A G, Vasileva Y N, Semenov D S, Kudryavtsev N D, Panina O Y, Khoruzhaya A N, Zinchenko V V, Akhmad E S, Petraikin A V, Vladzymyrskyy A V, Midaev A V, Morozov S P. “Chest MRI of patients with COVID-19.” Magn Reson Imaging. 2021 June; 79:13-19. doi: 10.1016/j.mri.2021.03.005. Epub 2021 Mar. 13. PMID: 33727149; PMCID: PMC7955570. In the study by Vasilev et al., all patients underwent magnetic resonance imaging (MRI) examinations using MR-LUND PROTOCOL: Single-shot Fast Spin Echo (SSFSE), LAVA 3D and IDEAL 3D, Echo-planar imaging (EPI) diffusion-weighted imaging (DWI) and Fast Spin Echo (FSE) T2 weighted imaging (T2WI).

Although the American College of Radiology suggested that the use of MRI be severely limited during the pandemic due to concerns over transmission of the virus, other MRI facilities suggested that patients in need of an MRI for its conventional uses not delay obtaining such MRI, and noted the extraordinary sanitization steps being taken to protect against spread of the virus. Further, the U.S. Food and Drug Administration (FDA) issued important guidelines recommending that patients undergoing MRI during the pandemic wear a non-metallic mask.

Viruses are typically composed of the following three parts providing it with its structure: a nucleic acid genome (genetic material, e.g., RNA), a protein which encases the nucleic acid and aids in the virus' replication inside a host cell, and a fatty outer layer of lipids. However, there are no covalent bonds involved, but rather, the viral self-assembly is based on weak “non-covalent” interactions between the proteins, RNA and lipids. The coronavirus comprises weak non-covalent bonds where Van der Waals forces weakly assist in holding it together. Van der Waals forces are weak intermolecular forces caused by attractions between very small dipoles in molecules. Polar molecules have permanent dipole-dipole interactions, and non-polar molecules can interact by way of Van der Waals (or London dispersion) forces.

Virus inactivation in vitro by radiation therapy (radiotherapy) is a known technique, the required dose being of the order of the Mrad, i.e., 10 kGy, but this treatment method could cause further complications if directed to a patient's lung already damaged by the COVID-19 infection, and further would be difficult to target the virus, or if a large area of the lungs is affected, would likewise be difficult to treat. It has further been suggested that rotating magnetic fields could potentially be applied in COVID-19 antiviral treatments. For example, Shallcross, U.S. Patent Application Publication No. 2006/0049110 teaches a device and method for purifying water and other substrates, such as surgical instruments, from pathogens, such as, bacteria, viruses, fungi, parasites and worms, utilizing at least one magnet mounted in rows on the faces of a block for rotation on a shaft, and a means to rotate the shaft, the method of treatment being carried out by rotating the shaft and bringing the object to be treated in close proximity with the magnets. Further, Liang, U.S. Patent Application Publication 20190240498 presents methods for targeting and killing types of cells or organisms using Magneto-Electric Nano-Particles under the control of an external magnetic field. A method was also presented for using Magneto-Electric Nano-Particles to stimulate or rejuvenate cells under an external magnetic field. One aspect of the Jiang's work provides a method for disrupting, or killing bacteria or viruses comprising (i) coating, binding or conjugating Magneto-Electric Nano-Particles (MENPs) with a guiding agent that specifically binds to a targeted bacterium or virus to produce Guiding-Agent-Conjugated MENPs (GAC-MENPs), (ii) binding the targeted bacteria or viruses with the GAC-MENPs, and (iii) applying a magnetic field to the GAC-MENPs to generate an electric field for disrupting or killing the targeted bacteria or viruses.

It has also been reported that exposure to oscillating magnetic fields has potential for the treatment of malaria by damaging the malaria parasite. See Harrill, R., UW News, Mar. 3, 2000, Magnetic fields may hold key to malaria treatment, UW researchers find, www.washington.edu/news/2000/03/30/magnetic-fields-may-hold-key-to-malaria-treatment-uw-researchers-find/, last accessed 7/3/2021. It has further been reported that the cutaneous leishmaniasis (CL) parasite can potentially be treated by having iron nanoparticles taken up by macrophages in the host, and then subjecting the nanoparticles to oscillating magnetic fields to cause localized heat to destroy the parasite. See Price, H., BMC, Can we treat leishmaniasis with tiny magnets? (Mar. 22, 2019), https://blogs. biomedcentral.com/bugbitten/2019/03/22/can-we-treat-leishmaniasis-with-tiny-magnets/—Last accessed 7/3/2021.

In the current methodology, a patient with an infection, such as the COVID-19 infection (or other pathogenic infection or malady), could be treated via exposure to magnetic fields, such as a pulsed magnetic field, a rotating magnetic field or the magnetic field present in an MRI, etc., for various treatment periods. In one embodiment, for example, the MRI would first be used in a conventional manner as a diagnostic tool to locate the source of the infection, e.g., in one or more of the patient's lungs, in the brain, cardiovascular areas, eyes, kidneys, etc. as has been used in the past. If a patient has already been diagnosed as being infected with the pathogen, and is suffering respiratory distress (in one or both lungs), then the use of the MRI treatment could begin on the affected lung(s) without the need to first run a diagnostic MRI. The diagnostic capabilities of the MRI could also be used secondarily to monitor the effectiveness of the MRI treatments on, e.g., reducing the area of infection (with other diagnostic tools being used to test for positive presence of the infection). In one embodiment, the patient is treated with a conventional MRI unit. In another embodiment, the patient is treated with a point-of-care (POC) MRI that can be readily transported to the patient's bedside.

Additionally, guided ultrasound procedures noted above could be employed in tandem with MRI, where the MRI is used not only to provide imaging of the tissues being treated, but can also be used itself to treat the infected tissue. Thus, the hybrid approach of MRI-guided ultrasound (cavitation) procedures could cause a dramatic disruption of the virus functionality (e.g., disruption of its outer membrane).

Additionally, although the treatment methodologies noted herein have focused on the treatment of patients with COVID-19 infections and the side effects thereof (post-COVID syndrome or long-haul COVID-19), these treatment methodologies and systems have application for use in the treatment of wounds or other illnesses. For example, aircraft-based HBOT treatment units can treat many patients at a time. Thus, it is envisioned that these mobile units could be deployed anywhere in the world for use in treating large numbers of patients desiring or requiring HBOT treatment for any particular disease. This is particularly advantageous with respect to simultaneous treatment of a large number of patients that are not presently harboring an infectious disease, thereby providing mass treatment capabilities without enhanced risk of spreading an infection within the treatment unit.

The above methods directed to treating humans (one type of subject patient) afflicted with a pathogenic disease could likewise be employed to treat other subject patients, namely, animals, plants, bee hives (pollinating bees), and the like suffering from a pathogenic disease. It will be understood that although the treatment methods herein have been described for use with human patients, where applicable, it is envisioned that such methods could be used on other subject patients, such as animals, livestock, beneficial insects, e.g., bees, and plant life as well. Furthermore, it will likewise be understood that the above methods can be used alone or in combination with other of the above methods. For example, treatment of pathogenic disease with ultrasonic cavitation or ultrasound can also be combined with magnetic field therapies, hyperbaric chamber use as described herein, and/or HBOT therapies. Also, a hyperbaric chamber or a pressurized airplane cabin, can be employed with combinations of fluctuating temperature, humidity and/or pressure to weaken or disable the pathogen, and can further be combined with ultrasonic/ultrasound and/or magnetic field therapies. As yet another example, treatment of pathogenic disease with magnetic field therapy can also be combined with the use of ultrasound/ultrasonic cavitation and/or the use of a hyperbaric chamber as described herein to create pressure differentials, etc.

All references referred to herein are incorporated herein by reference. While the apparatus, systems and methods of the present disclosure have been described in terms of preferred or illustrative embodiments, it will be apparent to those of skill in the art that variations may be applied to the processes and systems described herein without departing from the concept and scope of the invention. All such similar substitutes and modifications apparent to those skilled in the art are deemed to be within the scope and concept of the invention. Those skilled in the art will recognize that the methods and apparatus of the present invention has many applications, and that the present invention is not limited to the representative examples disclosed herein. Moreover, the scope of the present invention covers conventionally known variations and modifications to the system components described herein, as would be known by those skilled in the art.

We claim:

1. A method of treating a subject patient afflicted with a pathogenic disease caused by a pathogen, the pathogen comprising an outer membrane structure maintaining an internal pressure inside of the membrane, the method comprising the steps of:
a. placing the patient in a hyperbaric chamber capable of being pressurized or depressurized to a desired internal chamber pressure;
b. administering oxygen in the chamber to a desired level to enhance oxygenation levels within the human; and
c. adjusting the internal chamber pressure sufficient to cause a pressure differential between the pathogen internal pressure and the internal chamber pressure to cause the outer membrane of the pathogen to rupture or to otherwise disable the pathogen.

2. The method of claim 1 wherein the pathogen is selected from the group consisting of viruses, bacteria, fungi, parasites, worms, protozoa and helminths.

3. The method of claim 1 wherein the subject patient is a human being afflicted with a viral infection caused by a novel coronavirus pathogen and its variants.

4. The method of claim 3 wherein the novel coronavirus pathogen is SARS-CoV-2 and its variants and the human being is suffering from COVID-19.

5. The method of claim 1 wherein the step of adjusting the internal chamber pressure is achieved by increasing the internal chamber pressure.

6. The method of claim 5 wherein the pressure is increased between about 1.0 atm up to about 7 atm according to the tolerances of the subject patient being infected with the pathogen.

7. The method of claim 1 wherein the step of adjusting the internal chamber pressure is achieved by decreasing the internal chamber pressure.

8. The method of claim 7 wherein the pressure is decreased to about 0.5 atm or less according to the tolerances of the subject patient being infected with the pathogen.

9. The method of claim 1 wherein a series of pressure adjustments are employed.

10. The method of claim 9 wherein the series of pressure adjustments comprise incremental step pressure increases.

11. The method of claim 9 wherein the series of pressure adjustments comprise incremental step pressure decreases.

12. The method of claim 9 wherein the series of pressure adjustments comprise cycling between incremental step pressure increases and incremental step pressure decreases.

13. The method of claim 1 wherein the hyperbaric chamber is a single occupant unit.

14. The method of claim 1 wherein the hyperbaric chamber is a multiple occupant unit capable of treating multiple subject patients.

15. The method of claim 1 wherein the hyperbaric chamber comprises a pressurized full body suit worn by the subject patient.

16. The method of claim 1 wherein the hyperbaric chamber comprises an aircraft cabin capable of being pressurized.

17. The method of claim 1 further comprising the step of administering breathing assistance to the subject patient.

18. The method of claim 1 further comprising the step of administering oxygen to the subject patient at a constant desired concentration or at intervals of 100% oxygen followed by intervals of lower levels of oxygen.

19. The method of claim 1 further comprising the step of performing ultrasonic cavitation on a region of the subject patient.

20. The method of claim 1 further comprising the step of applying magnetic fields to a region of the subject patient.

21. The method of claim 1 further comprising one or more of the following steps:
a. varying the temperature in the chamber;
b. varying the humidity in the chamber; and/or
c. infusing additional gases into the chamber or directly into a part of the body of the human being at constant or varied concentrations, the additional gasses selected from the group consisting of: known respiratory inhaler gases such as those used in the treatment of asthma and COPD, e.g., corticosteroid inhalers (such as fluticasone), and bronchodilator inhalers (such as albuterol); inhalers infused with nasal decongestants (including natural menthol and camphor based products); ethyl alcohol vapor; oxygen; natural inhalable substances, e.g., herbal inhalers, aromatherapeutics; and treatment gases used for clearing mucus plugs in the human being's lungs.

22. The method of claim 21 further comprising the application of step pressure changes, magnetic fields, ultrasonic fields, or combinations thereof.

23. The method of claim 1 wherein the subject patient is afflicted with a bacterial infection.

24. The method of claim 3 wherein the human being is suffering from post COVID-19 syndrome.

25. The method of claim 1 wherein the subject patient is selected from the group consisting of humans, animals, livestock, plants and beneficial insects.

26. A mobile method of treating one or more human patients afflicted with a pathogenic disease caused by a pathogen, the pathogen comprising an outer structural membrane maintaining an internal pressure inside of the membrane, the mobile method comprising the steps of:
a. placing the one or more human patients in an air transport vehicle located on the ground, the air transport vehicle capable of obtaining in-flight altitudes where the external air pressure ranges between about 0.3-0.2 atm, the air transport vehicle further comprising:
i. a pressurizable fuselage integral to the air transport vehicle, the fuselage having climate control and oxygen supply;
ii. one or more independently pressurizable treatment chambers located within the fuselage of the air transport vehicle, the one or more pressurizable treatment chambers further comprising
climate control independent of the fuselage,
pressure control independent of the fuselage,
oxygen supply independent of the fuselage,
one or more seats, beds or treatment tables for receiving and securing the one or more human patients, and
medical supplies for maintaining the life of the one or more human patients;
iii. controllers for adjusting the climate in the one or more pressurizable treatment chambers; and
iv. controllers for adjusting the pressure in the one or more pressurizable treatment chambers;
b. flying the air transport vehicle to an altitude having a desired ambient external air pressure;
c. independently adjusting the air pressure in the one or more independently pressurizable treatment chambers to a desired treatment pressure sufficient to cause a pressure differential between the pathogen internal pressure and the internal chamber pressure to cause the outer membrane of the pathogen to rupture or to otherwise disable the pathogen;
d. maintaining the desired treatment pressure for a desired length of time; and
e. returning the air transport vehicle to the ground.

27. The mobile method of claim 26 further comprising the steps of varying the pressure in the one or more treatment chambers while the air transport vehicle is in flight to induce differential pressures sufficient to rupture or disrupt the pathogen membrane.

28. The mobile method of claim 26 further comprising the step of administering breathing assistance to the one or more human patients.

29. The mobile method of claim 26 wherein the one or more human patients are afflicted with a viral infection caused by a novel coronavirus pathogen and its variants.

30. The mobile method of claim 29 wherein the novel coronavirus is SARS-CoV-2 (and its variants) and the one or more human patients are suffering from COVID-19.

31. The mobile method of claim 26 wherein the step of independently adjusting the air pressure in the one or more independently pressurizable treatment chambers is achieved by increasing the air pressure in the one or more independently pressurizable treatment chambers.

32. The mobile method of claim 31 wherein the pressure is increased up to about 7 atm or to an upper limit value permitted in the air transport vehicle.

33. The mobile method of claim 26 wherein the step of independently adjusting the air pressure in the one or more independently pressurizable treatment chambers is achieved by decreasing the air pressure in the one or more independently pressurizable treatment chambers.

34. The mobile method of claim 31 wherein the pressure is decreased to about 0.5 atm or to a lower limit value permitted in the air transport vehicle or as can be tolerated by the one or more human patients.

35. The mobile method of claim 26 wherein a series of pressure adjustments are employed.

36. The mobile method of claim 35 wherein the series of pressure adjustments comprise incremental step pressure increases.

37. The mobile method of claim 35 wherein the series of pressure adjustments comprise incremental step pressure decreases.

38. The mobile method of claim 35 wherein the series of pressure adjustments comprise cycling between incremental step pressure increases and incremental step pressure decreases.

39. The mobile method of claim 26 wherein the one or more human patients are suffering from post COVID-19 syndrome.

40. A method of treating one or more subject patients afflicted with a pathogenic disease caused by a pathogen, the pathogen comprising an outer membrane maintaining an internal pressure inside of the membrane, the method comprising the steps of:
a. placing the one or more subject patients in an air transport vehicle located on the ground, the air transport vehicle further comprising:
i. a pressurizable fuselage integral to the air transport vehicle, the fuselage having climate control and oxygen supply;
ii. one or more a pressurizable treatment chambers located within the fuselage of the air transport vehicle, the one or more pressurizable treatment chamber further comprising
climate control independent of the fuselage,
pressure control independent of the fuselage,
oxygen supply independent of the fuselage,
a seat/bed/table for securing one or more human patients, and
medical supplies for maintaining the life of the one or more human patients;
iii. controllers for adjusting the climate in the one or more pressurizable treatment chambers; and
iv. controllers for adjusting the climate in the one or more pressurizable treatment chambers;
b. pressurizing the one or more treatment chambers to a desired pressure sufficient to cause a pressure differential between the pathogen internal pressure and the internal chamber pressure to cause the outer membrane of the pathogen to rupture or to otherwise disable the pathogen; and
c. returning the pressure in the one or more treatment chambers to an ambient pressure.

41. The method of claim 40 wherein the one or more subject patients are human patients afflicted with a viral infection caused by a novel coronavirus pathogen and its variants.

42. The method of claim 41 wherein the novel coronavirus is SARS-CoV-2 (and its variants) and the one or more human patients are suffering from COVID-19.

43. The method of claim 40 wherein the treatment chambers are beds.

44. The method of claim 40 wherein the treatment chambers are existing air transport vehicle seats.

45. The method of claim 40 wherein the treatment chambers are pressurized by the pressurizable fuselage.

46. The method of claim 40 wherein the one or more human patients are suffering from post COVID-19 syndrome.

47. A mobile treatment unit for treating one or more human patients afflicted with a pathogenic disease caused by a pathogen, the pathogen comprising an outer membrane maintaining an internal pressure inside of the membrane, the mobile treatment unit comprising:
a. an air transport vehicle capable of flying to altitudes where the external air pressure ranges between about 0.3-0.2 atm;
b. a pressurizable fuselage integral to the air transport vehicle, the fuselage having climate control and oxygen supply;
c. one or more independently pressurizable treatment chambers located within the fuselage of the air transport vehicle, the one or more pressurizable treatment chamber further comprising
i. climate control independent of the fuselage,
ii. pressure control independent of the fuselage,
iii. oxygen supply independent of the fuselage,
iv. one or more seats, beds or treatment tables for receiving and securing the one or more human patients, and
v. medical supplies for maintaining the life of the one or more human patients;
d. controllers for adjusting the climate in the one or more pressurizable treatment chambers; and
e. controllers for adjusting the pressure in the one or more pressurizable treatment chambers.

48. A nonsurgical method of destroying a pathogen present in a subject patient comprising the steps of:
a. identifying a body region of the subject patient afflicted with the pathogen; and
b. performing an ultrasonic cavitation procedure on an outer surface of the afflicted body region of the subject patient.

49. The nonsurgical method of claim 48 comprising the further steps of:
a. before the step of performing the ultrasonic cavitation, placing the subject patient in a hyperbaric chamber capable of being pressurized or depressurized to a desired internal chamber pressure; and
b. adjusting the internal chamber pressure to the desired internal chamber pressure.

50. The nonsurgical method of claim 48 comprising the further steps of using a guided ultrasound procedure in tandem with Magnetic Resonance Imaging.

51. The nonsurgical method of claim 48 wherein the pathogens are selected from the group consisting of viruses, bacteria, fungi, protozoa, helminths and parasites.

52. The nonsurgical method of claim 48 wherein the subject patient is a human patient afflicted with a viral infection caused by a novel coronavirus pathogen or its variants.

53. The nonsurgical method of claim 52 wherein the novel coronavirus is SARS-CoV-2 (and its variants) and the human patient is suffering from COVID-19.

54. The method of claim 53 wherein the human patient is suffering from post COVID-19 syndrome.

55. The nonsurgical method of claim 49 further comprising providing hyperbaric oxygen treatment.

56. A nonsurgical method of destroying pathogens present in an anatomical location of a subject patient, the method comprising the steps of:
a. identifying the anatomical location of the subject patient where the pathogen is present;
b. performing an ultrasonic procedure on a region of an outer surface of the subject patient proximate the identified anatomical location to direct ultrasonic forces toward the pathogens in such anatomical location.

57. The nonsurgical method of claim 56 comprising the further step of using a guided ultrasound procedure in tandem with Magnetic Resonance Imaging.

58. The nonsurgical method of claim 56 comprising the further step of carrying out the method within a hyperbaric chamber while also providing hyperbaric oxygen treatment to the subject patient.

59. The nonsurgical method of claim 56 wherein the pathogens are selected from the group consisting of viruses, bacteria, fungi, protozoa, helminths and parasites.

60. The nonsurgical method of claim 56 wherein the subject patient is a human patient afflicted with a viral infection caused by a novel coronavirus pathogen or its variants.

61. The nonsurgical method of claim 60 wherein the novel coronavirus is SARS-CoV-2 (and its variants) and the human patient is suffering from COVID-19.

62. The nonsurgical method of claim 60 wherein the human patient is suffering from post COVID-19 syndrome.

63. A nonsurgical method of destroying pathogens present in an anatomical location of a subject patient, the method comprising the steps of:
a. identifying the anatomical location of the subject patient where the pathogens are present;
b. applying magnetic field forces on a region of an outer surface of the subject patient proximate the identified anatomical location to direct the magnetic field forces toward the pathogens in such anatomical location.

64. The nonsurgical method of claim 63 wherein the magnetic field force is selected from the group consisting of Magnetic Resonance Imaging magnetic fields, pulsed magnetic fields, rotating magnetic fields, alternating magnetic fields, oscillating magnetic fields, dynamic magnetic fields, magnetic nanoparticles, and magnetic hyperthermia.

65. A nonsurgical method of destroying fat cells in a human or animal patient comprising the steps of:
a. placing the human or animal patient in a hyperbaric chamber capable of being pressurized or depressurized to a desired internal chamber pressure;
b. adjusting the internal chamber pressure to the desired internal chamber pressure; and
c. performing an ultrasonic cavitation procedure on a desired portion of an outer surface of the human or animal patient where the fat cell destruction is desired.

66. The nonsurgical method of claim 65 further comprising the step of providing hyperbaric oxygen treatment.

67. An enhanced method of fatty tissue removal in a human being, the fatty tissue comprising one or more adipose cells each having a generally spherical membrane surrounding a fat reservoir, the fat reservoir having an internal fat reservoir pressure, the method comprising the steps of:
a. placing the patient in a hyperbaric chamber capable of being pressurized or depressurized to a desired internal chamber pressure;
b. adjusting the internal chamber pressure sufficient to cause a pressure differential between the fat reservoir internal pressure and the internal chamber pressure to place stress on the one or more adipose cell generally spherical membranes; and
c. performing an ultrasonic cavitation procedure on a desired portion of an outer surface of the human where the fat cell destruction is desired.

68. The enhanced method of claim 67 further comprising the step of providing hyperbaric oxygen treatment.

69. A method of disinfecting medical equipment contaminated with a pathogen, the pathogen comprising an outer membrane maintaining an internal pressure inside of the membrane, the method comprising the steps of:
a. placing the equipment in a hyperbaric chamber capable of being pressurized or depressurized to a desired internal chamber pressure; and
b. adjusting the internal chamber pressure sufficient to cause a pressure differential between the pathogen internal pressure and the internal chamber pressure to cause the outer membrane of the pathogen to rupture or to otherwise disable the pathogen.

70. The method of claim 69 wherein the desired internal chamber pressure ranges between 0.0 atm to 100 atm excluding 1 atm.

71. The method of claim 69 wherein the step of adjusting the internal chamber pressure is carried out by rapidly increasing or decreasing the internal chamber pressure.

72. The method of claim 69 wherein the step of adjusting the internal chamber pressure is carried out by cycling between increasing and decreasing the internal chamber pressure.

73. The method of claim 69 wherein the pathogens are selected from the group consisting of viruses, bacteria, fungi, protozoa, helminths and parasites.

74. The method of claim 69 wherein the pathogen is a novel coronavirus pathogen and its variants.
